(19) United States
(12) Patent Application Publication
(10) Pub. No.: US 2008/0262554 A1
(43) Pub. Date: Oct. 23, 2008

(54) Dyanamic rod

(76) Inventors: Stanley Kyle Hayes, Mission Viejo, CA (US); Joey Camia Reglos, Lake Forest, CA (US); Moti Altarac, Irvine, CA (US); Daniel H. Kim, Houston, TX (US)

Correspondence Address: RIMAS LUKAS;VERTIFLEX, INC., 1351 CALLE AVANZADO, SAN CLEMENTE, CA, 92673, US

(21) Appl. No.: 12/154,540

(22) Filed: May 23, 2008

Related U.S. Application Data
(63) Continuation-in-part of application No. 11/427,738, filed on Jun. 29, 2006.
Application No. 11/427,738 is a continuation-in-part of application No. 11/436,407, filed on May 17, 2006.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
(60) Provisional application No. 60/931,811, filed on May 25, 2007.

(51) Int. Cl. A61B 17/70

(52) U.S. Cl. 606/278; 606/257

(57) ABSTRACT

A dynamic rod implantable into a patient and connectable between two vertebral anchors in adjacent vertebral bodies is provided. The dynamic rod fixes the adjacent vertebral bodies together in a dynamic fashion providing immediate postoperative stability and support of the spine. The dynamic rod comprises a first rod portion having a first engaging portion and a second rod portion having a second engaging portion. The first and second rod portions are connected to each other at the first and second engaging portions. The dynamic rod further includes at least one bias element configured to provide a bias force in response to deflection or translation of the first rod portion relative to the second rod portion. The dynamic rod permits relative movement of the first and second rod portions allowing the rod to carry some of the natural flexion and extension moments that the spine is subjected to.

### CROSS-REFERENCE TO RELATED APPLICATIONS

This application claims priority to and the benefit of U.S. Provisional Patent Application Ser. No. 60/931,811 entitled “Dynamic Rod” filed on May 25, 2007 which is incorporated herein by reference in its entirety. This application also claims priority to and is a continuation-in-part of co-pending U.S. patent application Ser. No. 11/427,738 entitled “Systems and methods for stabilization of the bone structures” filed on Jun. 29, 2006 which is a continuation-in-part of U.S. patent application Ser. No. 11/436,407 entitled “Systems and methods for stabilization of the bone structures” filed on May 17, 2006 which is a continuation-in-part of U.S. patent application Ser. No. 11/033,452 entitled “Systems and methods for stabilization of the bone structures” filed on Jan. 10, 2005 which is a continuation-in-part of U.S. patent application Ser. No. 11/006,495 entitled “Systems and methods for stabilization of the bone structures” filed on Dec. 6, 2004 which is a continuation-in-part of U.S. patent application Ser. No. 10/970,366 entitled “Systems and methods for stabilization of the bone structures” filed on Oct. 20, 2004. The referenced applications are each incorporated herein by reference in their entirety.

### FIELD

The present invention generally relates to devices, systems, and methods for the fixation of the spine. In particular, the present invention relates to a system applied posteriorly to the spine that provides dynamic support to spinal vertebrae and controls load transfers to avoid deterioration of the bone of adjacent spinal vertebrae.

### BACKGROUND

Damage to the spine as a result of advancing age, disease, and injury, has been treated in many instances by fixation or stabilization of vertebrae. Conventional methods of spinal fixation utilize a rigid spinal fixation device to support an injured spinal vertebra relative to an adjacent vertebra and prevent movement of the injured vertebra relative to an adjacent vertebra. These conventional spinal fixation devices include anchor members for fixing to a series of vertebrae of the spine and at least one rigid link element designed to interconnect the anchor members. Typically, the anchor member is a screw and the rigid link element is a rod. The screw is configured to be inserted into the pedicle of a vertebra to a predetermined depth and angle. One end of the rigid link element is connected to an anchor inserted in the pedicle of the upper vertebra and the other end of the rod is connected to an anchor inserted in the pedicle of an adjacent lower vertebra. The rod ends are connected to the anchors via coupling constructs such that the adjacent vertebrae are supported and held apart in a relatively fixed position by the rods. Typically two rods and two pairs of anchors are installed each in the manner described above such that two rods are employed to fix two adjacent vertebrae, with one rod positioned on each side of adjacent vertebrae. Once the system has been assembled and fixed to a series of two or more vertebrae, it constitutes a rigid device preventing the vertebrae from moving relative to one another. This rigidity enables the devices to support all or part of the stresses instead of the stresses being born by the series of damaged vertebra.

While these conventional procedures and devices have been proven capable of providing reliable fixation of the spine, the resulting constructs typically provide a very high degree of rigidity to the operative levels of the spine resulting in decreased mobility of the patient. Unfortunately, this high degree of rigidity imparted to the spine by such devices can sometimes be excessive. Because the patient's fixed vertebrae are not allowed to move, the vertebrae located adjacent to, above or below, the series that has undergone such fixation tend to move more in order to compensate for the decreased mobility. As a result, a concentration of additional mechanical stresses is placed on these adjacent vertebral levels and a sharp discontinuity in the distribution of stresses along the spine can then arise between, for example, the last vertebra of the series and the first free vertebra. This increase in stress can accelerate degeneration of the vertebrae at these adjacent levels.

Sometimes, fixation accompanies a fusion procedure in which bone growth is encouraged to bridge the intervertebral body disc space to thereby fuse adjacent vertebrae together. Fusion involves removal of a damaged intervertebral disc and introduction of an interbody spacer along with bone graft material into the intervertebral disc space. In cases where fixation accompanies fusion, excessively rigid spinal fixation is not helpful to the promotion of the fusion process due to load shielding away from the fixed series. Without the stresses and strains, bone does not have loads to adapt to and as bone loads decrease, the bone becomes weaker. Thus, fixation devices that permit load sharing and assist the bone fusion process are desired in cases where fusion accompanies fixation.

Various improvements to fixation devices such as a link element having a dynamic central portion have been devised. These types of dynamic rods support part of the stresses and help relieve the vertebrae that are overtaxed by fixation. Some dynamic rods are designed to permit axial load transmission substantially along the vertical axis of the spine to prevent load shielding and promote the fusion process. Dynamic rods may also permit a bending moment to be partially transferred by the rod to the fixed series that would otherwise be born by vertebrae adjacent to the fixed series. Compression or extension springs can be coiled around the rod for the purpose of providing de-rotation forces as well as relative translational sliding movement along the vertical axis of the spine. Overall, the dynamic rod in the fixation system plays an important role in recreating the biomechanical organization of the functional unit made up of two fixed vertebrae together with the intervertebral disc.

In conclusion, conventional spinal fixation devices have not provided a comprehensive solution to the problems associated with curing spinal diseases in part due to the difficulty of creating a system that mimics a healthy functioning spinal unit. Hence, there is a need for an improved dynamic spinal fixation device that provides a desired level of flexibility to the fixed series of the spinal column, while also providing long-term durability and consistent stabilization of the spinal column.

### SUMMARY

According to one aspect of the invention, a dynamic rod is provided. The dynamic rod includes a first rod portion and a second rod portion. The first rod portion has a first engaging portion at one end. The first engaging portion has a second rod receiving portion configured to receive the second rod portion. The first engaging portion further has a first bias element receiving portion. The second rod portion has a second engaging portion at one end. The second engaging portion has a second bias element receiving portion. The first and second rod portions are connected to each other at the first and second engaging portions such that at least a portion of the second engaging portion is disposed in the second rod receiving portion. The dynamic rod further includes a retainer configured to keep the first and second rod portions together and at least a first bias element configured to provide a bias force. At least a portion of the first bias element is disposed in the first bias element receiving portion and at least another portion of the first bias element is disposed in the second bias element receiving portion. The first bias element is disposed between the first and second rod portions. In one variation, the first bias element receiving portion is located inside the second rod receiving portion. In another variation, the retainer is configured to encompass at least a portion of the first rod portion and at least a portion of the second rod portion and connected to the first rod portion such that the second rod portion is capable of movement relative to the first rod portion. In another variation, the dynamic further includes a stiffener located between the first and second rod portions. In yet another variation, the dynamic rod further includes a second bias element wherein the second rod engaging portion includes a flange and the retainer includes a interior ledge and the second bias element is disposed between the flange and the ledge. In another variation of the invention, the bias element is configured to provide a bias force on one of the first and second rod portions relative to the other of the first and second rod portions. In another variation of the invention, the bias element is configured to provide a bias force on one of the first and second rod portions upon relative motion with respect to the other of the first and second rod portions.

According to another aspect of the invention, a dynamic rod having a first rod portion and a second rod portion is provided. The first rod portion has a first engaging portion at one end. The first engaging portion has a first bias element receiving portion. The second rod portion has a second engaging portion at one end. The second engaging portion has a second bias element receiving portion. The first and second rod portions are connected to each other at the first and second engaging portions. The dynamic rod further includes a retainer configured to keep the first and second rod portions together and at least a first bias element configured to provide a bias force. At least a portion of the first bias element is disposed in the first bias element receiving portion and at least another portion of the first bias element is disposed in the second bias element receiving portion. The first bias element is disposed between the first and second rod portions. In one variation, the retainer is configured to encompass the first bias element. In another variation, the dynamic rod further includes a bearing element disposed between the first and second engaging portions. In another variation, the first engaging portion overlaps the second engaging portion such that a cross-section of the first engaging portion taken perpendicular to the longitudinal axis of the dynamic rod is complementary to the second engaging portion at said cross-section. In another variation, the first and second engaging portions have thread-like grooves configured to receive a coil-like first bias element. In another variation, the dynamic rod further includes at least one second bias element. In another variation, the second bias element is substantially circular in shape with a central aperture for receiving a rod portion therein with the first or second rod portion located in the central aperture and the second bias element further includes a plurality of slits that open at the outer periphery of the bias element and extend inwardly toward the longitudinal axis of the dynamic rod. In another variation, the second bias element is ring-like in shape and includes a central aperture for receiving a rod portion therein with the first or second rod portion located in the central portion and an opening in the second bias element forming two fingers that constrict the central aperture. In another variation, the at least a first bias element is configured to provide a bias force on one of the first and second rod portions relative to the other of the first and second rod portions. In another variation, the at least a first bias element is configured to provide a bias force on one of the first and second rod portions relative to the other of the first and second rod portions upon motion of one of the first and second rod portions with respect to the other one of the first and second rod portions.

According to another aspect of the invention, a dynamic rod having a first rod portion and a second rod portion is provided. The first rod portion has a first engaging portion at one end. The first engaging portion has a second rod receiving portion configured to receive a second rod portion. The second rod portion has a shaped second engaging portion at one end. The first and second rod portions are connected to each other at the first and second engaging portions such that the second engaging portion is disposed in the second rod receiving portion and such that the first rod portion is movable relative to the second rod portion. The dynamic rod further includes a retainer configured to keep the first and second rod portions together and at least a first bias element configured to provide a bias force. The first bias element is disposed in the second rod receiving portion between the shaped second engaging portion and the retainer. In one variation, the second rod receiving portion is a bore having a partially spherical shaped bottom and the second engaging portion has a partially spherical shape corresponding to the partially spherical shaped bottom such that the second engaging portion moves relative to the base to pivot the second rod portion relative to the first rod portion. In another variation, the second rod receiving portion is a bore having a base and the base includes a raised portion configured to contact the second engaging portion such that the second engaging portion pivots about the contact. In another variation, the second bias element disposed between the base and the second engaging portion. In another variation, the bias element is configured to provide a bias force on one of the first and second rod portions with respect to the other of the first and second rod portions upon motion of one of the first and second rod portions with respect to the other one of the first and second rod portions. In one variation, the at least a first bias element is configured to provide a bias force on one of the first and second rod portions with respect to the other of the first and second rod portions.

According to another aspect of the invention, a dynamic rod having a first rod portion and a second rod portion is provided. The first rod portion has a first engaging portion at one end. The second rod portion has a second engaging portion at one end. The first and second rod portions are connected to each other at the first and second engaging portions such that the first rod portion is movable relative to the second rod portion. The dynamic rod further includes at least a first bias element configured to provide a bias force on one of the first and second rod portions upon relative motion with respect to the other of the first and second rod portions. At least a portion of the first bias element is disposed between the first and second rod portions and the first bias element includes a central opening and at least partially encompasses one of the first and second rod portions. The first bias element includes a radial axis that is not constant. In one variation, the first bias element includes a major axis and a minor axis and the first bias element is closer to one of the first and second rod portions at the minor axis and closer to the other of the first and second rod portions at the major axis. In another variation, the non-constant radial axis forms a plurality of corrugations in the first bias element. In another variation, the bias element includes at least one at least partially encompassing component. In another variation, the encompassing component includes at least one landing perpendicular to the longitudinal axis of the dynamic rod. In another variation, the bias element includes a plurality of stacked encompassing components.

According to another aspect of the invention, a dynamic rod having a first rod portion and a second rod portion is provided. The first rod portion has a first engaging portion at one end. The second rod portion has a second engaging portion at one end. The first and second rod portions are connected to each other at the first and second engaging portions such that the first and second engaging portions form at least one overlap configured to impart the dynamic rod with greater flexibility at intersection of the first and second engaging portions relative to the rest of the rod portions such that the first rod portion is movable relative to the second rod portion. In one variation, the first and second rod portions are integrally formed from the same piece. In another variation, the at least one overlap forms at least one interdigitation of first and second rod portions. In another variation, the dynamic rod further includes a retainer configured to connect the first and second rod portions together.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates an exploded perspective view of a dynamic rod according to the present invention.

FIG. 2 illustrates a side view of a dynamic rod of FIG. 1 according to the present invention.

FIG. 3 illustrates a cross-sectional view of a first rod portion of the dynamic rod of FIG. 1 according to the present invention.

FIG. 4 illustrates a cross-sectional view of a second rod portion of the dynamic rod of FIG. 1 according to the present invention.

FIG. 5 illustrates a bias element of the dynamic rod of FIG. 1 according to the present invention.

FIG. 6 illustrates a perspective view of a retainer of the dynamic rod of FIG. 1 according to the present invention.

FIG. 7a illustrates a perspective view of another variation of a dynamic rod according to the present invention.

FIG. 7b illustrates an exploded view of the dynamic rod of FIG. 7a according to the present invention.

FIG. 8a illustrates a side view of a dynamic rod in a contracted state according to the present invention.

FIG. 8b illustrates a side view of a dynamic rod in an extended state according to the present invention.

FIG. 8c illustrates a side view of a dynamic rod in an extended and deflected state according to the present invention.

FIG. 8d illustrates a side view of a dynamic rod in a contracted and deflected state according to the present invention.

FIG. 9a illustrates a perspective view of another variation of the dynamic rod according to the present invention.

FIG. 9b illustrates an exploded view of the dynamic rod of FIG. 9a according to the present invention.

FIG. 9c illustrates a cross-sectional view of the retainer of the dynamic rod of

FIGS. 9a and 9b according to the present invention.

FIG. 10a illustrates a perspective view of another variation of a dynamic rod according to the present invention.

FIG. 10b illustrates an exploded view of the dynamic rod of FIG. 10a according to the present invention.

FIG. 10c illustrates a bias element according to the present invention.

FIG. 10d illustrates the bias element of FIG. 10c disposed within a retainer according to the present invention.

FIG. 10e illustrates the bias element of FIG. 10c disposed within a dynamic rod according to the present invention.

FIG. 11a illustrates a partially transparent side view of another variation of a dynamic rod according to the present invention.

FIG. 11b illustrates a cross-sectional view of the dynamic rod of FIG. 11a according to the present invention.

FIG. 11c illustrates a partially exploded view of the dynamic rod of FIG. 11c according to the present invention.

FIG. 12a illustrates a perspective view of a bias element according to the present invention.

FIG. 12b illustrates a top view of the bias element of FIG. 12a according to the present invention.

FIG. 13a illustrates a perspective view of a bias element according to the present invention.

FIG. 13b illustrates a top view of the bias element of FIG. 13a according to the present invention.

FIG. 13c illustrates a cross-sectional view of the bias element of FIG. 13b according to the present invention.

FIG. 13d illustrates a perspective view of a bias element according to the present invention.

FIG. 13e illustrates a side view of the bias element of FIG. 13d according to the present invention.

FIG. 13f illustrates a top view of the bias element of FIG. 13d according to the present invention.

FIG. 13g illustrates a perspective view of the bias element of FIG. 13f according to the present invention.

FIG. 14a illustrates a perspective view of a bias element according to the present invention.

FIG. 14b illustrates a top view of the bias element of FIG. 14a according to the present invention.

FIG. 15a illustrates a perspective view of a bias element according to the present invention.

FIG. 15b illustrates a top view of the bias element of FIG. 15a according to the present invention.

FIG. 16a illustrates a partially transparent side view of a dynamic rod according to the present invention.

FIG. 16b illustrates an exploded view of the dynamic rod of FIG. 16a according to the present invention.

FIG. 16c illustrates a cross sectional view of the dynamic rod of FIG. 16a according to the present invention.

FIG. 17a illustrates a perspective view of a dynamic rod according to the present invention.

FIG. 17b illustrates a cross-sectional view of the dynamic rod of FIG. 17a according to the present invention.

FIG. 17c illustrates a perspective view of a variation of the dynamic rod of

FIG. 17a according to the present invention.

FIG. 17d illustrates a perspective view of the dynamic rod of FIG. 17c deployed within anchors according to the present invention.

FIG. 18a illustrates a perspective view of a variation of a dynamic rod according to the present invention.

FIG. 18b illustrates a perspective view of the dynamic rod of FIG. 18a without a retainer according to the present invention.

FIG. 18c illustrates a top view of a bias element employed in the dynamic rod of FIG. 18a according to the present invention.

FIG. 18d illustrates a cross-sectional view of the dynamic rod of FIG. 18e illustrating another variation of a bias element according to the present invention.

FIG. 18e illustrates a perspective view of a dynamic rod without a retainer according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants.

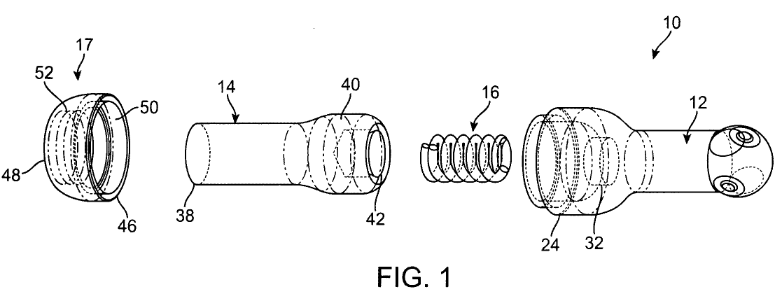

Referring now to FIGS. 1-7, there is shown a dynamic rod 10 for use in a spinal fixation system. A spinal fixation system generally includes a first set of two bone anchor systems installed into the pedicles of a superior vertebral segment, a second set of two bone anchor systems installed into the pedicles of an inferior vertebral segment, a first link element connected between one of the pedicle bone anchor systems in the first set and one of the pedicle bone anchor systems in the second set along the same side of the inferior and superior vertebral segments, and a second link element connected between the other of the pedicle bone anchor systems in the first set and the other of the pedicle bone anchor systems in the second set along the same side of the inferior and superior vertebral segments.

A typical anchor system comprises, but is not limited to, a spinal bone screw that is designed to have one end that inserts threadably into a vertebra and a seat at the opposite end thereof. Typically, the seat is designed to receive the link element in a channel in the seat. The link element is typically a rod or rod-like member. The seat typically has two upstanding arms that are on opposite sides of the channel that receives the rod member. The rod is laid in the open channel, the top of which is then closed with a closure member to both capture the rod in the channel and lock it in the seat to prevent relative movement between the seat and the rod.

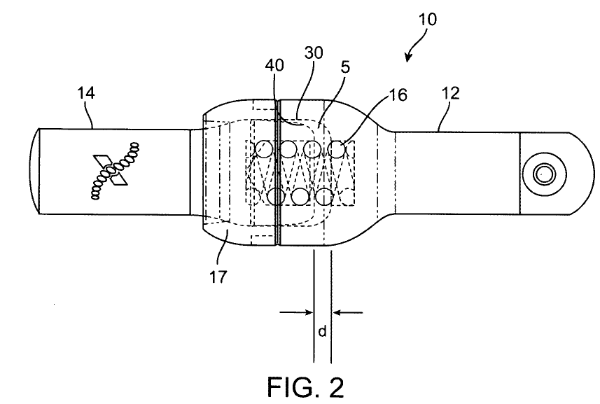

With particular reference to FIGS. 1 and 2, a rod 10 according to the present invention comprises a first rod portion 12, a second rod portion 14, a bias element 16, and a retainer 17 or other connecting means. The first rod portion 12 is connected to the second rod portion 14 via the retainer 17. The bias element 16 is disposed within and between the first and second rod portions 12, 14 as shown in FIG. 2.

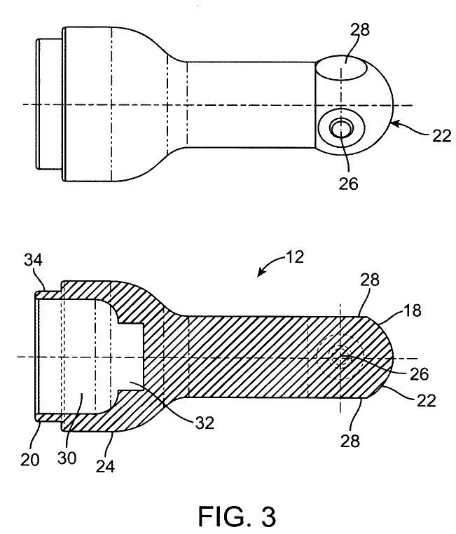

Referring now to FIG. 3, the first rod portion 12 includes a first end 18 and a second end 20. The first rod portion 12 is generally cylindrical, elongate and rod-like in shape. An anchor connecting portion 22 is formed at the first end 18 and configured for attachment to an anchor system. The anchor connecting portion 22 shown in FIG. 3 is partially spherical in shape and includes oppositely disposed outwardly extending pins 26 for engaging slots or apertures formed in the anchor to allow the dynamic rod 10 to pivot about the pins 26 when connected to the anchor. The anchor connecting portion 22 also includes oppositely disposed flat areas 28. When the dynamic rod 10 is connected to the anchor and pivoted into a substantially horizontal position, the flat areas 28 face upwardly and downwardly and as a result, provide a lower profile for the rod within the seat of the anchor. Furthermore, the flat areas 28 provide a flat contact surface for a closure member on the upper surface of the rod and a flat contact surface on the bottom surface when seated in the anchor. Although FIG. 3 shows the rod having an anchor connecting portion 22 configured for a pin-to-slot engagement, the invention is not so limited and any suitable anchor connecting portion configuration is within the scope of the present invention.

Still referencing FIG. 3, the first rod portion 12 includes an engaging portion 24 at a slightly enlarged and bulbous second end 20. The engaging portion 24 is configured to engage the second rod portion 14 of the dynamic rod 10. The engaging portion 24 includes a first bore defining a receiving portion 30 for receiving the second rod portion 14. The engaging portion 24 also includes a second bore concentrically aligned with and formed within the first bore defining a bias element receiving portion 32. A collar 34 is also formed at the second end 20 that is configured to mate with the retainer 17. The collar 34 has a slightly smaller outer diameter than the rest of the bulbous engaging portion 20. With the retainer 17 mated with the male member collar 34, the intersection of the first rod portion 12 and retainer 17 is flush.

Turning now to FIG. 4, there is shown a second rod portion 14. The second rod portion 14 includes a first end 36 and a second end 38. The second rod portion 14 is generally cylindrical, elongate and rod-like in shape and includes an engaging portion 40 at a slightly enlarged and bulbous first end 36. The engaging portion 40 is configured to engage with the first rod portion 12 of the dynamic rod 10. The engaging portion 40 of the second rod portion 14 further includes a first bore defining a bias element receiving portion 42 for receiving the bias element 16 therein. At least a portion of the engaging portion 40 of the second rod portion 14 is configured and sized to fit within the receiving portion 30 of the first rod portion 14 as shown in FIG. 2. The outer surface of the engaging portion 40 is tapered such that the engaging portion narrows towards the first end 36. In one variation, the slope of the outer surface is approximately three degrees with respect to the longitudinal axis of the second rod portion 14; however, the invention is not so limited and the slope is selected for customizing the angulation of the second rod portion 14 relative to the first rod portion 12 when connected therewith. The second rod portion 14 further includes a beveled first end 36 having a radius of curvature of approximately 0.063 millimeters; however, the invention is not so limited and any suitable radius of curvature or none at all is within the scope of the present invention. The bevel is located closer to the first end 36 relative to the taper. The bevel also plays a role in permitting the second rod portion 14 to angulate when disposed inside the first rod portion 12.

The second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, may include a pin-and-slot or other configuration such as that shown in FIG. 3 for the anchor connecting portion 22 of the first rod portion 12.

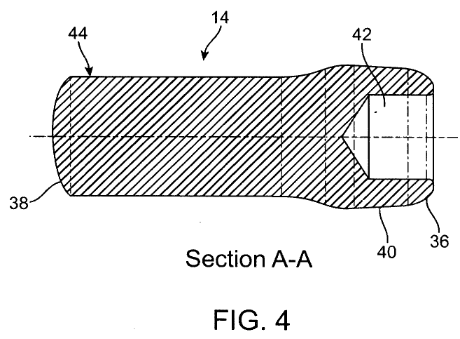

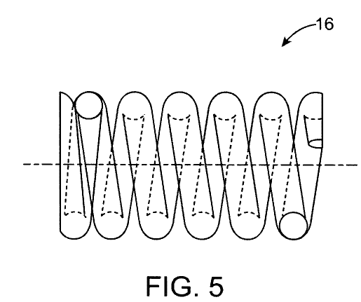

Referring now to FIG. 5, there is shown a bias element 16 according to the present invention. In the variation shown, the bias element 16 is a coil or spring. The bias element 16 is made from any suitable material such as titanium or PEEK. The bias element 16 is sized to be receiving inside the bias element receiving portion 32 of the first rod portion 12 and the bias element receiving portion 42 of the second rod portion 14. Although a coiled spring is shown in FIG. 5, the invention is not so limited and any suitable type of bias element may be employed. Different types of biasing elements will be discussed in greater detail below.

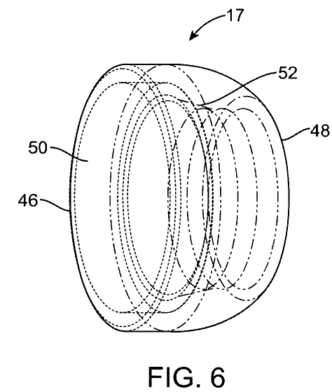

Turning now to FIG. 6, there is shown a retainer 17 having a first end 46 and a second end 48 according to the present invention. The retainer 17 is generally cylindrical and sleeve-like in shape and has a bore opening to and extending between the first and second ends 46, 48. The retainer 17 is configured to encompass at least a portion of the first rod portion 12 and at least a portion of the second rod portion 14. Accordingly, the bore defines a first receiving portion 50 at the first end 46 configured to receive therein at least a portion of the first rod portion 12 and, in particular, configured to receive the collar 34 of the first rod portion 12 as shown in FIG. 2. The bore also defines a second receiving portion 52 at the second end 48 that is configured to receive therein at least a portion of the second rod portion 14 and, in particular, configured to receive at least a portion of the engaging portion 40 of the second rod portion 14. The retainer 17 forms a constriction such that the second end 48 has a smaller diameter relative to the diameter of the retainer at the first end 46. The interior surface of the retainer 17 substantially corresponds to the geometry being received within the retainer 17.

Referring back to FIGS. 1 and 2, the assembly of the dynamic rod 10 will now be discussed. The bias element 16 is placed inside the bias element receiving portion 42 of the second rod portion 14. The second rod portion 14 together with the bias element 16 is connected to the first rod portion 12 by pushing the bias element 16 into the bias element receiving portion 32 of the first rod portion 12 and pushing the engaging portion 40 of the second rod portion 14 into the engaging portion 24 of the first rod portion 12. The second end 38 of the second rod portion 14 is then inserted into the first end 46 of the retainer 17 and passed through the second end 48 such that the collar 34 of the first rod portion 12 is disposed inside the first receiving portion 50 of the retainer 17 and at least a portion of the engaging portion 40 of the second rod portion 14 is disposed inside the second receiving portion 52 of the retainer 17. The retainer 17 is connected to the first rod portion 12 by a laser weld or an e-beam weld or other suitable means such that the second rod portion 14 is captured by the retainer 17 constriction and retained within the retainer 17 and the first rod portion 12 such that the second rod portion 14 is capable of movement relative to the retainer 17 and the first rod portion 12. In particular, the second rod portion 14 is capable of displacement from the longitudinal axis and/or movement along the longitudinal axis relative to the retainer 17 and the first rod portion 12 and/or rotation about the longitudinal axis. As shown in FIG. 2, the second rod portion 14 when fully extended from the first rod portion 12, defines a distance “d” between the first end 36 of the second rod portion 14 and the end wall of the rod engaging portion 24. This distance “d” defines in part the extent of movement along the longitudinal axis of the second rod portion 14 relative to the first rod portion 12 as well as the degree of displacement of the second rod portion 14 relative to the longitudinal axis that is permitted by the configuration. In one variation, the distance “d” is approximately one or two millimeters; however, the invention is not so limited and the distance “d” may be selected to be any suitable distance. FIG. 2 also shows the space “s” between the interior surface of the rod receiving portion 30 and the tapered and beveled surfaces of the engaging portion 40 of the second rod portion 14. Space “s” also defines in part the extent of movement along the longitudinal axis of the second rod portion 14 relative to the first rod portion 12 as well as the degree of displacement of the second rod portion 14 relative to the longitudinal axis that is permitted by the configuration.

After the dynamic rod 10 is assembled, it is ready to be implanted within a patient and be connected to anchors planted in pedicles of adjacent vertebral bodies preferably in a manner such that the first rod portion 12 of the dynamic rod 10 illustrated in FIGS. 1-6 is oriented cephalad and connected to the upper anchor and the second rod portion 14 is placed caudad and connected to the lower anchor. Because the first rod portion 12 includes an anchor connecting portion 22 configured such that connection with the anchor does not result in the rod extending cephalad beyond the anchor, this orientation and configuration of the dynamic rod is advantageous particularly because it avoids impingement of adjacent anatomy in flexion or in extension of the patient.

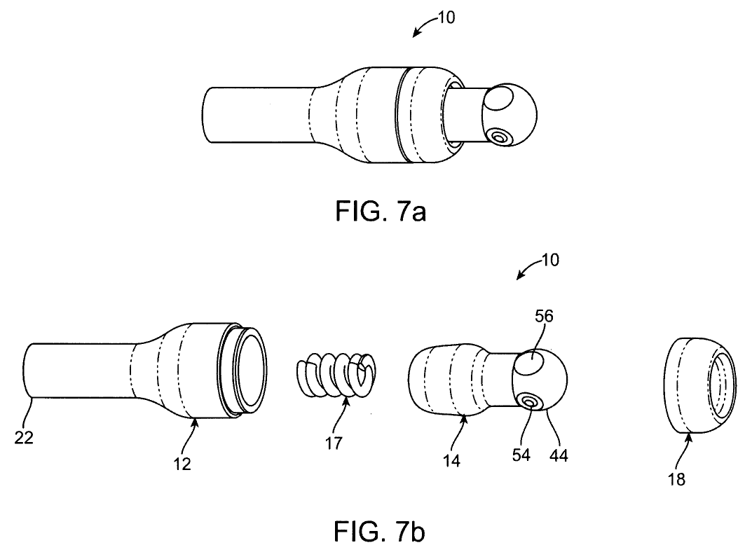

In an alternative variation shown in FIGS. 7a and 7b, the dynamic rod 10 is implanted into the patient such that the first rod portion 12 is oriented caudad and the second rod portion 14 is oriented cephalad. As shown in FIGS. 7a and 7b, the second rod portion 14 includes an anchor connecting portion 44 that is partially spherical in shape and includes oppositely disposed outwardly extending pins 54 for engaging slots or apertures formed in the upper anchor to allow the dynamic rod 10 to pivot about pins 54 when connected to the anchor. The anchor connecting portion 44 also includes oppositely disposed flat areas 56 as described above. The second rod portion 14 of the dynamic rod 10 illustrated in FIG. 7 is oriented cephalad and connected to the upper anchor and the first rod portion 12 is placed caudad and connected to the lower anchor. Because the second rod portion 14 includes an anchor connecting portion 44 configured such that connection with the anchor does not result in excessive rod extending cephalad beyond the anchor, this orientation and configuration of the dynamic rod is advantageous particularly because it avoids impingement of adjacent anatomy in flexion or in extension of the patient.

Therefore, it is noted that the preferred implantation method and preferred orientation of the dynamic rod 10 is such that there is minimal or substantially no “overhanging” rod that extends cephalad beyond the upper anchor. Such orientation is achieved by the orientation of the rod during implantation as well as by the configuration of the anchor connecting portion 22, 44 of either one or both of the first rod portion 12 and second rod portion 14 such that the anchor connecting portion 22, 44 is configured such that there is substantially no overhang beyond the anchor.

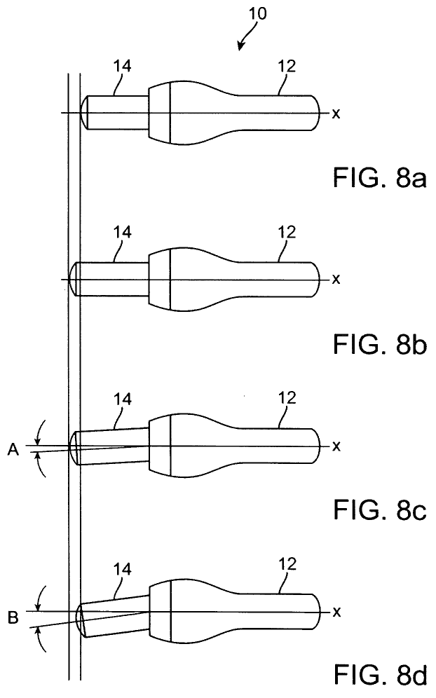

The implanted dynamic rod and anchor system fixes the adjacent vertebral bodies together in a dynamic fashion providing immediate postoperative stability and support of the spine. Referring now to FIG. 8, the dynamic features of the dynamic rod 10 according to the present invention will now be discussed. In FIG. 8a, there is shown a dynamic rod 10 according to the present invention with the second rod portion 14 completely pushed within the first rod portion 12. FIG. 8b shows the second rod portion 14 extended along the longitudinal axis “x” relative to the first rod portion 12. As described above, the degree of longitudinal extension is determined by the configuration of the first and second rod portions 12, 14 and is approximately between zero and five millimeters, preferably approximately one millimeter; however, the invention is not so limited and any suitable longitudinal extension is within the scope of the present invention. FIG. 8c illustrates displacement or angulation from the longitudinal axis of the second rod portion 14 relative to the first rod portion 14 by an angle “A” while the second rod portion 14 is also longitudinally in extension relative to the first rod portion 12. Angle “A” is approximately between zero and five degrees, preferably approximately three degrees with respect to the longitudinal axis “x”. FIG. 8d shows the second rod portion 14 displaced from the longitudinal axis “x” by an angle “B” and extended longitudinally. Angle “B” is approximately between zero and five degrees, preferably approximately three degrees with respect to the longitudinal axis “x”.

Hence, FIG. 8 illustrates that the dynamic rod allows for movement described by a displacement from the longitudinal axis as well as movement along the longitudinal axis alone or in combination allowing the rod to carry some of the natural flexion and extension moments that the spine is subjected to. In cases where the dynamic rod is subjected to a force displacing one of the rod portions relative to the other rod portion away from the longitudinal axis, at least a portion of the bias element 16 is also displaced from the longitudinal axis. The resulting displacement of the bias element 16 from the longitudinal axis establishes a biasing force exerted by the bias element in a direction opposite to its displacement to force the displaced rod portion back into a normal “relaxed” position substantially aligned with the longitudinal axis. Substantial polyaxial rotation of the second rod portion relative to the first rod portion is within the scope of motion of the dynamic rod.

In one variation, the bias element 16 is a compression spring that becomes shorter when axially loaded and acts as an extension mechanism such that when disposed in the assembled dynamic rod 10 and axially loaded, the bias element 16 exerts a biasing force pushing the first rod portion 12 and the second rod portion 14 apart. In one variation, the bias element 16 is configured such that it exerts a biasing force pushing the first rod portion 12 and second rod portion 14 apart by the maximum degree permitted by the dynamic rod configuration such that when longitudinally loaded the second rod portion 14 will move inwardly towards the first rod portion 12 and the bias element will tend to push the second rod portion 14 outwardly relative to the first rod portion 12.

In another variation, the bias element 16 is a tension spring that becomes longer when axially loaded and acts as a contraction mechanism such that when disposed in the assembled dynamic rod 10 and axially loaded, the bias element 16 exerts a biasing force pulling the first rod portion 12 and the second rod portion 14 together. In this variation, the dynamic rod 10 under load is advantageously permitted to elongate; and when elongated, the bias element 16 urges the rod 10 to its contracted static length and not shorter than the static length thereby maintaining the desired minimum distraction distance.

In another variation, the bias element 16 is a coil configured to not exhibit spring-like characteristics when loaded along the longitudinal axis. Instead, the coil serves a stabilizer for loads having a lateral force component, in which case the lateral biasing is provided by the bias element.

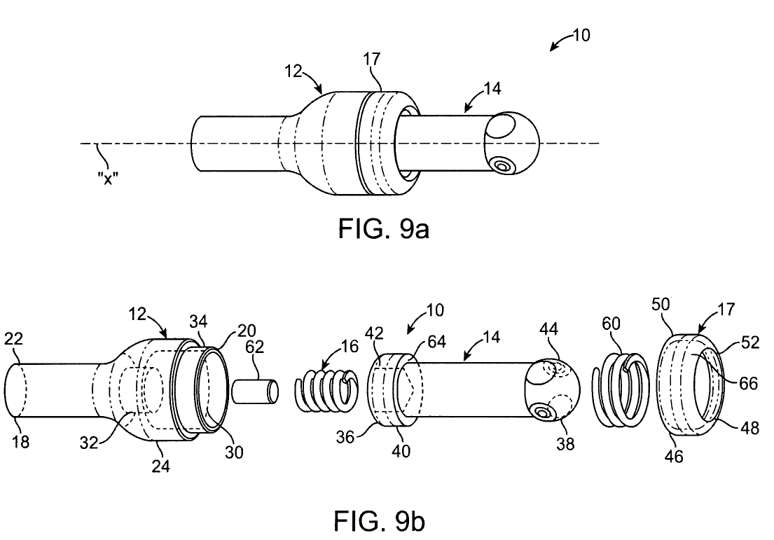

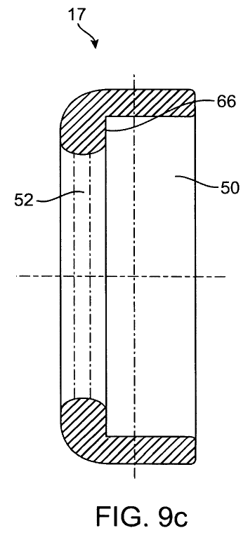

Another dynamic rod 10 according to the present invention is shown in FIGS. 9a and 9b wherein like numbers are used to describe like parts herein. In this variation, in addition to the first rod portion 12, second rod portion 14, a bias element 16, and a retainer 17 or other connecting means, there is a second bias element 60 and an optional stiffener 62. The first rod portion 12 is connected to the second rod portion 14 via the retainer 17 and the first bias element 16 is disposed within and between the first and second rod portions 12, 14. The second bias element 60 is disposed between the retainer 17 and second rod portion 14.

Still referencing FIGS. 9a and 9b and with particular reference to FIG. 9b, the first rod portion 12 includes an engaging portion 24 at a slightly enlarged and bulbous second end 20. The engaging portion 24 is configured to engage the second rod portion 14 of the dynamic rod 10. The engaging portion 24 includes a first bore defining a receiving portion 30 for receiving the second rod portion 14. The engaging portion 24 also includes a second bore concentrically aligned with and formed within the first bore defining a bias element receiving portion 32. A collar 34 is also formed at the second end 20 which is configured to mate with the retainer 17. The collar 34 has a slightly smaller diameter than the rest of the bulbous engaging portion 20. With the retainer 17 mated with the male member collar 34, the intersection of the first rod portion 12 and retainer 17 is flush at the outer surface. The first end 18 of the first rod portion 12 includes an anchor connecting portion 22 configured to be connected to an anchor. The anchor connecting portion 22 is sized and configured to be seated in a channel of a seat of a bone screw anchor for example. Any configuration for the second end 18 that is suitable for connection to an anchor is within the scope of the present invention and, for example, may include a pin-and-slot or other configuration such as that shown in FIG. 3 for the anchor connecting portion 22 of the first rod portion 12.

With particular reference to FIG. 9b, the second rod portion 14 includes a first end 36 and a second end 38. The second rod portion 14 is generally cylindrical, elongate and rod-like in shape and includes an engaging portion 40 at an enlarged first end 36. The engaging portion 40 is configured to engage with the first rod portion 12 of the dynamic rod 10. The engaging portion 40 of the second rod portion 14 further includes a first bore defining a bias element receiving portion 42 for receiving the bias element 16 therein. At least a portion of the engaging portion 40 of the second rod portion 14 is configured and sized to fit within the receiving portion 30 of the first rod portion 14 as shown in FIG. 9a. In this variation, the engaging portion 40 includes an encompassing shoulder or flange 64 that extends outwardly from at least a portion of the central portion of the second rod portion 14. The shoulder 64 is configured as an abutment for the second bias element 60. The outer surface of the engaging portion 40 is tapered such that the engaging portion narrows towards the first end 36. In one variation, the slope of the outer surface is approximately three degrees with respect to the longitudinal axis of the second rod portion 14; however, the invention is not so limited and the slope can is selected for customizing the angulation of the second rod portion 14 relative to the first rod portion 12. The second rod portion 14 further includes a beveled first end 36 having a radius of curvature of approximately 0.063 millimeters; however, the invention is not so limited and any suitable radius of curvature or none at all is within the scope of the present invention. The bevel is located closer to the first end 36 relative to the taper. Both the taper and the bevel play a role in permitting the second rod portion 14 to angulate with respect to the first rod portion 12 when disposed inside the receiving portion 30.

Still referencing FIG. 9b, the second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, includes the pin-and-slot style configuration as shown in FIG. 9b and discussed above.

Still referencing FIG. 9b, the bias element 16 is made from any suitable material such as titanium or PEEK. The bias element 16 is sized to be receiving inside the bias element receiving portion 32 of the first rod portion 12 and the bias element receiving portion 42 of the second rod portion 14. Although a coiled spring is shown in FIG. 5 as the bias element, the invention is not so limited and any suitable type of bias element may be employed.

Still referencing FIG. 9b, one variation includes a stiffener 62 that is substantially cylindrical and made of any suitable material such as titanium or PEEK. The stiffener 62 is sized to fit within the bias element 16, that is, the stiffener is sized to fit inside the coils of the spring 16. Furthermore, the stiffener 62 is long enough to reach into both the bias element receiving portion 32 in the first rod portion 12 and the bias element receiving portion 42 along with the bias element 16 when the first and second rod portions 12, 14 are assembled. The stiffener 62 provides additional rigidity to the dynamic rod 10 when it is subject to deflection from the longitudinal axis “x”. The stiffener 62 is also employed to customize the degree of translation along the longitudinal axis and to serve as a stop. For example, a longer stiffener 62 reduces the distance which the first rod portion 12 can move in the longitudinal direction relative to the second rod portion 14. Likewise, a shorter stiffener 62 increases the travel distance along the longitudinal axis of the first rod portion 12 relative to the second rod portion 14. The stiffener 62 is optional and may be excluded from the embodiment shown in FIG. 9a and 9b.

Still referencing FIG. 9b, there is shown a retainer 17 having a first end 46 and a second 48 according to the present invention. The retainer 17 is generally cylindrical in shape and has a bore opening to and extending between the first and second ends 46, 48. The retainer 17 is configured to encompass at least a portion of the first rod portion 12 and at least a portion of the second rod portion 14. Accordingly, the bore defines a first receiving portion 50 at the first end 46 configured to receive therein at least a portion of the first rod portion 12 and, in particular, configured to receive the collar 34 of the first rod portion 12. The bore also defines a second receiving portion 52 at the second end 48 that is configured to receive therein at least a portion of the second rod portion 14 and, in particular, configured to receive at least a portion of the central portion of the second rod portion 14 to capture the enlarged engaging portion 40 inside the retainer 17. To capture the engaging portion 40, the retainer 17 forms a constriction such that the second end 48 has a smaller diameter opening relative to the diameter of the opening at the first end 46. The interior surface of the retainer 17 substantially corresponds to the geometry being received within the retainer 17. In one variation, the intersection of the first receiving portion 50 and the second receiving portion 52 inside the retainer 17 forms an inner circumferential ledge 66 as shown in FIG. 9c. The inner circumferential ledge 66 serves as an abutment for the other end of the second bias element 60.

Still referencing FIG. 9b, there is shown a second bias element 60. The second bias element 60 is made from any suitable material such as titanium or PEEK. The second bias element 16 is sized to encompass the central portion or neck of the second rod portion 14 as well as to abut the shoulder 64 of the engaging portion 40 at one end and the circumferential ledge 66 at the other end of the second bias element 60. Although a coiled spring is shown in FIGS. 9a and 9b as the bias element, the invention is not so limited and any suitable type of bias element may be employed for the same function. Different types of biasing elements will be discussed in greater detail below.

Still referencing both FIGS. 9a and 9b, the assembly of the dynamic rod 10 will now be discussed. The first bias element 16 is placed inside the bias element receiving portion 42 of the second rod portion 14. The second rod portion 14 together with the first bias element 16 is connected to the first rod portion 12 by pushing the first bias element 16 into the bias element receiving portion 32 of the first rod portion 12 and pushing the engaging portion 40 of the second rod portion 14 into the rod receiving portion 30 of the first rod portion 12. The second bias element 60 is passed over the second end 38 and onto the central portion of the second rod portion 14 until it abuts the shoulder 64. The second end 38 of the second rod portion 14 is then inserted into the first end 46 of the retainer 17 and passed through the second end 48 of the retainer 17 such that the collar 34 of the first rod portion 12 is disposed inside the first receiving portion 50 of the retainer 17 and at least a portion of the central portion of the second rod portion 14 is disposed inside the second receiving portion 52 of the retainer 17. One end of the second bias element 60 abuts the inner circumferential ledge 66 of the retainer.

The retainer 17 is connected to the first rod portion 12 by a laser weld or an e-beam weld or other suitable means such that the second rod portion 14 is captured by the retainer 17 constriction and retained within the retainer 17 and first rod portion 12 such that the second rod portion 14 is capable of movement relative to the retainer 17 and the first rod portion 12. In particular, the second rod portion 14 is capable of rotation, displacement from the longitudinal axis and/or movement along the longitudinal axis relative to the retainer 17 and the first rod portion 12 such movement being biased by the first bias element 16 and the second bias element 60. Similar to the embodiment shown in FIG. 2, the second rod portion 14 when fully extended from the first rod portion 12, defines a distance “d” between the first end 36 of the second rod portion 14 and the bottom of the rod engaging portion 24. This distance “d” defines in part the extent of movement along the longitudinal axis of the second rod portion 14 relative to the first rod portion 12 as well as the degree of displacement of the second rod portion 14 relative to the longitudinal axis that is permitted by the configuration. In one variation, the distance “d” is approximately one or two millimeters; however, the invention is not so limited and the distance “d” may be selected to be any suitable distance. FIG. 2 also shows the space “s” between the interior surface of the rod receiving portion 30 and the tapered and beveled surfaces of the engaging portion 40 of the second rod portion 14. It is this space “s” that provides room for and defines the degree of deflection in part that the second rod portion 14 is capable of with respect to the first rod portion 12.

The dynamic rod 10 of FIGS. 9a and 9b is implanted into the patient in the same manner as described above with respect to the embodiments of FIGS. 1-8 and fixes the adjacent vertebral bodies together in a dynamic fashion. The dynamic rod assembly permits relative movement of the first and second rod portions 12, 14 providing immediate postoperative stability and support of the spine. The dynamic rod allows for polyaxial movement described by a displacement from the longitudinal axis as well as rotation and movement along the longitudinal axis alone or in combination allowing the rod to carry some of the natural flexion and extension moments that the spine is subjected to.

While the first bias element 16 provides the same dynamic response described above with respect to FIGS. 1-8, the stiffener, if employed, generally limits displacement and longitudinal movement of the first rod portion 12 relative to the second rod portions 14.

The second bias element 60 may be employed with or without the first bias element 16. In one variation, the second bias element 60 is a compression spring that becomes shorter when axially loaded and acts as an extension mechanism such that when disposed in the assembled dynamic rod 10 and axially loaded into compression, the second bias element 60 exerts a biasing force moving the second rod portion 14 and retainer 17 apart. When extended beyond the static length, the second bias element 60 exerts a biasing force towards the static length position. Such a configuration advantageously tends to return a contraction or extension of the rod to a normal static “relaxed” position. In this variation, the dynamic rod 10 under extension load is advantageously permitted to elongate; and when elongated, the bias element 16 urges the rod 10 back to its contracted static length thereby biasing the elongation inwardly.

In another variation, the bias element 60 is a tension spring that becomes longer when axially loaded and acts as a contraction mechanism such that when disposed in the assembled dynamic rod 10 and axially loaded, the bias element 60 exerts a biasing force pulling the second rod portion 12 and the retainer 17 together. The tension spring is incapable of being compressed due to its static closely coiled length. In this variation, the dynamic rod 10 under a negative compression load extends the second bias element 60; and when extended, the second bias element 60 urges the second rod portion 14 and retainer 17 together.

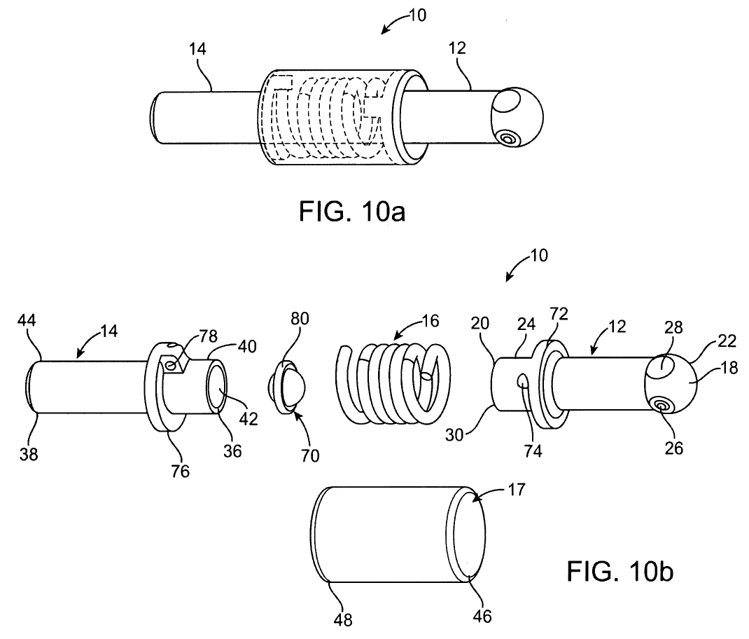

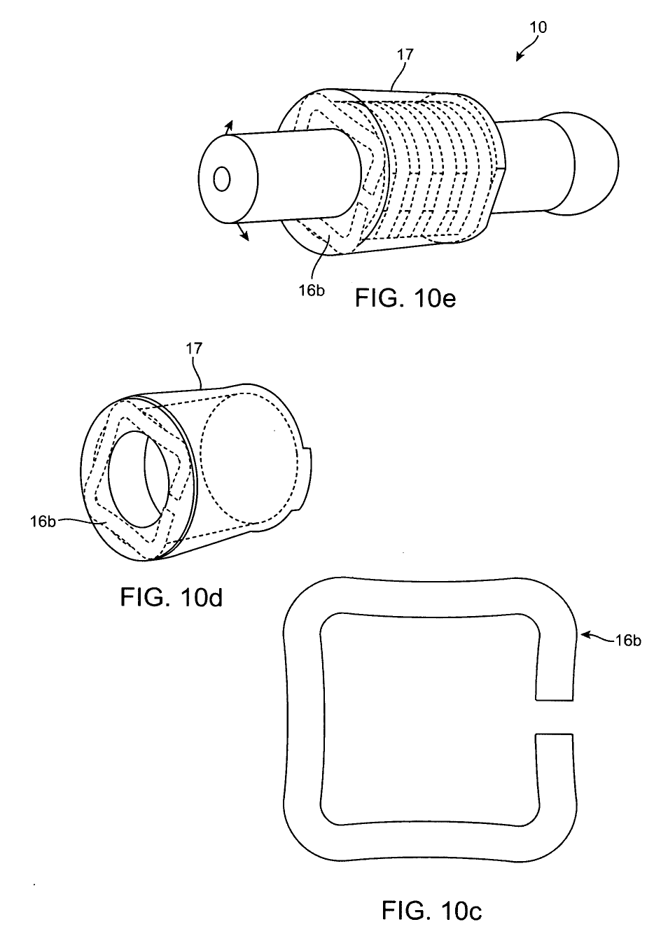

Turning now to FIGS. 10a and 10b, another dynamic rod 10 according to the present invention is depicted wherein like numbers are used to describe like parts herein. The dynamic rod 10 comprises a the first rod portion 12, second rod portion 14, a bias element 16, a retainer 17 or other connecting means, and a bearing element 70. The first rod portion 12 is connected to the second rod portion 14 via the bias element 16 with the bearing element 70 being disposed within and between the first and second rod portions 12, 14. The retainer 17 encompasses the joint, encasing the bias element 60, the bearing element 70 and portions of the first and second rod portions 12, 14.

With particular reference to FIG. 10b, the first rod portion 12 includes a first end 18 and a second end 20. The first rod portion 12 is generally cylindrical, elongate and rod-like in shape. An anchor connecting portion 22 is formed at the first end 18 and configured for attachment to an anchor system. The anchor connecting portion 22 shown in FIG. 10b is partially spherical in shape and includes oppositely disposed outwardly extending pins 26 for engaging slots or apertures formed in the anchor to allow the dynamic rod 10 to pivot about the pins 26 when connected to the anchor. The anchor connecting portion 22 also includes oppositely disposed flat areas 28. When the dynamic rod 10 is connected to the anchor and pivoted into a substantially horizontal position, the flat areas 28 face upwardly and downwardly and as a result, provide a lower profile for the rod within seat of the anchor. Furthermore, the flat areas 28 provide a flat contact surface for a closure member on the upper surface of the rod and a flat contact surface on the bottom surface when seated in the anchor. Although FIG. 10b shows the rod having an anchor connecting portion 22 configured for a pin-to-slot engagement, the invention is not so limited and any suitable anchor connecting portion configuration is within the scope of the present invention.

Still referencing FIG. 10b, the first rod portion 12 includes an engaging portion 24 at the second end 20. The engaging portion 24 is configured to engage the second rod portion 14 of the dynamic rod 10. The engaging portion 24 includes a recess conforming to at least a part of the shape of the bearing element 70 and defining a receiving portion 30 for bearing element 70. The first rod portion 12 includes an encompassing shoulder or flange 72 that extends outwardly from at least a portion of the first rod portion 12. The shoulder 72 is configured as an abutment for the bias element 16 and in one variation the shoulder 72 includes an integrally formed bias element receiving portion 74 for securing the bias element 16.

Still referencing FIG. 10b, there is shown a second rod portion 14. The second rod portion 14 includes a first end 36 and a second end 38. The second rod portion 14 is generally cylindrical, elongate and rod-like in shape and includes an engaging portion 40 at the first end 36. The engaging portion 40 is configured to engage with the first rod portion 12 of the dynamic rod 10. The engaging portion 40 of the second rod portion 14 includes a recess conforming to at least a part of the shape of the bearing element 70 and defining a receiving portion 42 for receiving the bearing element 70 therein. The second rod portion 14 includes a shoulder or flange 76 that extends outwardly from at least a portion of the second rod portion 14. The shoulder 76 is configured as an abutment for the bias element 16 and in one variation the shoulder 76 includes an integrally formed bias element receiving portion 78 for securing the bias element 16.

The second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, may include a pin-and-slot or other configuration such as that shown in FIG. 3 for the anchor connecting portion 22 of the first rod portion 12.

Still referencing FIG. 10b, there is shown a bias element 16 according to the present invention. In the variation shown, the bias element 16 is a spring or coil. The bias element 16 is made from any suitable material such as titanium or PEEK. The bias element 16 is sized to encompass at least a portion of the first and second rod portions 12, 14. In particular, the bias element 16 is sized to encompass engaging portions 24, 40 of the first and second rod portions 12, 14, respectively. Although a coiled spring is shown in FIG. 10b, the invention is not so limited and any suitable type of bias element may be employed.

Still referencing FIG. 10b, there is shown a bearing element 70. The bearing element 70 is configured and sized to fit at least partially within receiving portions 30, 42 of the first and second rod portions 12, 14, respectively. In one variation, the bearing element 70 is substantially spherical in shape serving as a spherical pivot and providing a bearing surface for the second rod portion 14 to angulate with respect to the first rod portion 12. The bearing element 70 is made from titanium anodized to create a lubricious surface to reduce wear. In one variation, the spherical bearing element 70 includes an outwardly extending circumferential flange 80. The flange 80 serves as a spacer as well as an abutment for the first and second rod portions 12, 14.

Still referencing FIG. 10b, there is shown a retainer 17 having a first end 46 and a second 48 according to the present invention. The retainer 17 is generally cylindrical in shape and has a bore opening to and extending between the first and second ends 46, 48. The retainer 17 is configured to encompass at least a portion of the first rod portion 12 and at least a portion of the second rod portion 14. The retainer 17 is made of titanium, PEEK, polyeurathane or silicone or any other suitable polymeric or metallic material. The retainer 17 may be injection molded around the dynamic rod 10 after it is assembled.

Referring back to FIGS. 10a and 10b, the assembly of the dynamic rod 10 will now be discussed. The bearing element 80 is disposed inside one of the receiving portions 30, 42 of the first and second rod portions 12, 14 and the bias element 16 is placed on one of the engaging portions 24, 40 and the first and second rod portions 12, 14 are brought together to capture the bearing element 70 inside recesses of each of the first and second rod portions 12, 14. One end of the bias element 16 engages the flange 72 of the first rod portion 12 and the other end of the bias element 16 engages the flange 76 of the second rod portion 14. Where bias element receiving portions 74, 78 are formed, the ends of the bias element 16 are engaged therewith and welded thereto. The retainer 17 is then installed. Alternatively, the retainer 17 may be installed on one of the rod portions 12, 14 prior to bringing the rod portions 12, 14 together. In general, the dynamic rod 10 is assembled such that rod portions 12, 14 are capable of relative movement with respect to each other.

In a variation shown in FIGS. 10c to 10e, a second bias element 16b is provided and disposed between the retainer 17 and flange 72 or flange 76 as shown in FIG. 10e. The second bias element 16b is substantially square with rounded corners; however, the invention is not so limited and any polygon or other shape may be employed for the second bias element 16b. In yet another variation, a third bias element may be disposed between the retainer and the other one of the flanges 72, 76. The second and third bias elements provide additional support and stability to the dynamic rod and serves as a bias for both motion of at least one rod portion along the longitudinal axis as well as for displacement of at least one rod portion from the longitudinal axis.

The dynamic rod 10 of FIGS. 10a to 10e is implanted into the patient in the same manner as described above with respect to the embodiments of FIGS. 1-9 and fixes the adjacent vertebral bodies together in a dynamic fashion. The dynamic rod assembly permits relative movement of the first and second rod portions 12, 14 providing immediate postoperative stability and dynamic support of the spine. The dynamic rod allows for rotation, displacement or angulation from the longitudinal axis of one rod portion relative to the other and/or movement along the longitudinal axis allowing the rod to carry some of the natural rotation, flexion and extension moments that the spine is subjected to. In cases where the dynamic rod is subjected to a force displacing one of the rod portions relative to the other rod portion away from the longitudinal axis, at least a portion of the bias element 16 is also displaced from the longitudinal axis. The resulting displacement of the bias element 16 from the longitudinal axis establishes a biasing force exerted by the bias element in a direction opposite to its displacement to force the displaced rod portion back into a position substantially aligned with the longitudinal axis.

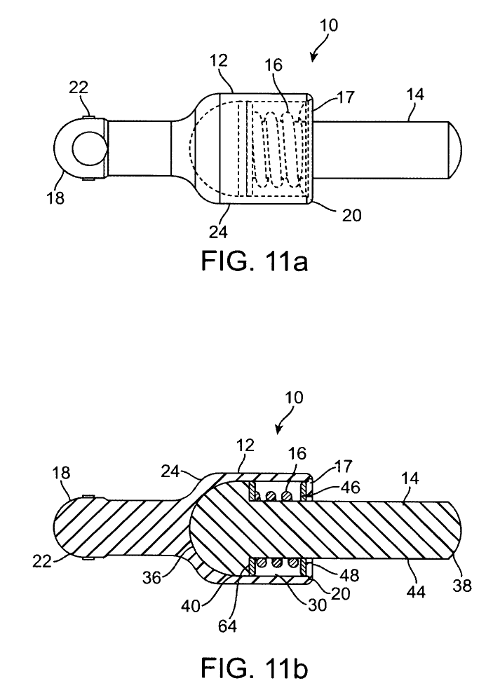

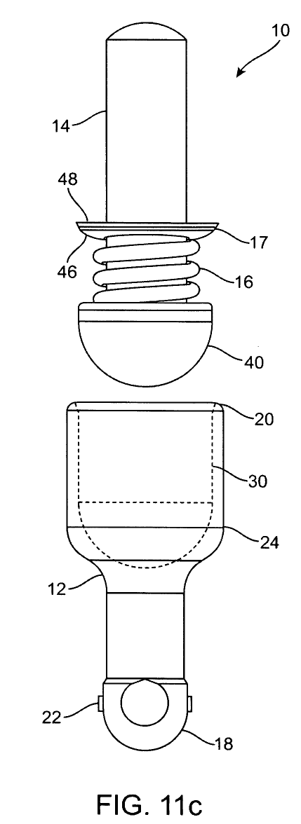

Another dynamic rod 10 according to the present invention is shown in FIGS. 11a, 11b and 11c wherein like numbers are used to describe like parts herein. In this variation, the dynamic rod 10 includes a first rod portion 12, second rod portion 14, at least one bias element 16, and a retainer 17 or other connecting means. The first rod portion 12 is connected to the second rod portion 14 via the retainer 17 and the first bias element 16 is disposed between the first and second rod portions 12, 14.

Still referencing FIGS. 11a and 11b and with particular reference to FIG. 11c, the first rod portion 12 includes an engaging portion 24 at a slightly enlarged and bulbous second end 20. The engaging portion 24 is configured to engage the second rod portion 14 of the dynamic rod 10. The engaging portion 24 includes a first bore defining a receiving portion 30 for receiving the second rod portion 14. The receiving portion 30 is shaped to complement the shape of the portion of the second rod portion 14 received therein. The second end 20 is configured to mate with the retainer 17. The first end 18 of the first rod portion 12 includes an anchor connecting portion 22 configured to be connected to an anchor. The anchor connecting portion 22 is sized and configured to be seated in a channel of a seat of a bone screw anchor for example. Any configuration for the second end 18 that is suitable for connection to an anchor is within the scope of the present invention and, for example, may include a pin-and-slot or other configuration such as that shown in FIG. 11c for the anchor connecting portion 22 of the first rod portion 12.

With particular reference to FIG. 11b, the second rod portion 14 includes a first end 36 and a second end 38. The second rod portion 14 is generally cylindrical, elongate and rod-like in shape and includes an engaging portion 40 at an enlarged first end 36. The engaging portion 40 is configured to engage with the first rod portion 12 of the dynamic rod 10. The engaging portion 40 of the second rod portion 14 is configured and sized to fit within the receiving portion 30 of the first rod portion 14 as shown in FIGS. 11a and 11b. In this variation, the engaging portion 40 includes an encompassing shoulder or flange 64 that extends outwardly from at least a portion of the central portion of the second rod portion 14. The shoulder 64 is configured as an abutment for the bias element 16. The rest of the engaging portion 40 forms a substantially semi-spherical or curved shape. The outer surface of the engaging portion 40 may be tapered such that the engaging portion narrows towards the second end 38. In one variation, the slope of the outer surface is approximately three degrees with respect to the longitudinal axis of the second rod portion 14; however, the invention is not so limited and the slope can is selected for customizing the angulation of the second rod portion 14 relative to the first rod portion 12. In addition, a bevel may be formed on the engaging portion 40 located closer to the second end 38. Both the taper and the bevel play a role in permitting the second rod portion 14 to angulate with respect to the first rod portion 12 when disposed inside the receiving portion 30.

Still referencing FIG. 11b, the second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, includes the pin-and-slot style configuration as shown with respect to the first rod portion 12 and discussed above.

Still referencing FIGS. 11a, 11b and 11c, the bias element 16 is made from any suitable material such as titanium or PEEK. The bias element 16 is sized to encompass at least a portion of the second rod portion 14 and to be received inside the rod receiving portion 30 of the first rod portion 12. Although a coiled spring is shown in FIG. 11 as the bias element, the invention is not so limited and any suitable type of bias element may be employed.

Still referencing FIGS. 11b and 11c, there is shown a retainer 17 having a first end 46 and a second 48 according to the present invention. In one variation, the retainer 17 is disc-like in shape and has a central bore opening to and extending between the first and second ends 46, 48 to allow passage for the central portion of the second portion 14 of the dynamic rod 10. The retainer 17 is configured to encompass at least a portion of the second rod portion 14. To capture the engaging portion 40, the retainer 17 forms a constriction such that the second end 20 has a smaller diameter opening thereby at least partially closing the bore opening at the second end 20 of the first rod portion 12. The first end 46 of the retainer 17 serves as an abutment for the bias element 16.

Still referencing both FIGS. 11a, 11b and 11c, the assembly of the dynamic rod 10 will now be discussed. The first bias element 16 is placed around the central portion of the second rod portion 14 such that it abuts the shoulder 64. The second rod portion 14 together with the first bias element 16 is inserted into the receiving portion 30 of the first rod portion 12. The curved shape of the engaging portion 40 is complemented by a curved shape of the end wall of the receiving portion 30. The complementary surfaces permit sliding engagement of the first and second rod portions 12, 14. The retainer 17 is passed over the second end 38 of the second rod portion 14 such that the bore of the retainer 17 receives the central portion of the second rod portion 14. The retainer 17 is connected by laser weld or other suitable attachment means to the first rod portion 12 at the second end capturing the engaging portion 40 inside the receiving portion 30 with the bias element 17 disposed between the retainer 17 and shoulder 64.

The engaging portion 40 of the second rod portion 14 is captured by the retainer 17 and contained within the retainer receiving portion 30 of the first rod portion 12 such that the second rod portion 14 is capable of movement relative to the retainer 17 and the first rod portion 12. In particular, the second rod portion 14 is capable of rotation, polyaxial displacement from the longitudinal axis and/or movement along the longitudinal axis relative to the retainer 17 and the first end portion 12 such movement being biased by the bias element 16. Similar to the embodiment shown in FIG. 2, the second rod portion 14 when fully extended from the first rod portion 12, defines a distance “d” between the first end 36 of the second rod portion 14 and the end of the receiving portion 30. This distance “d” defines in part the extent of movement along the longitudinal axis of the second rod portion 14 relative to the first rod portion 12 as well as the degree of displacement of the second rod portion 14 relative to the longitudinal axis that is permitted by the configuration. In one variation, the distance “d” is approximately one or two millimeters; however, the invention is not so limited and the distance “d” may be selected to be any suitable distance. Also similar to FIG. 2, the space “s” between the interior surface of the rod receiving portion 30 and the tapered and beveled surfaces of the engaging portion 40 of the second rod portion 14 provides room for and defines the degree of deflection that the second rod portion 14 is capable of with respect to the first rod portion 12.

The dynamic rod 10 of FIGS. 11a, 11b and 9c is implanted into the patient in the same manner as described above with respect to the embodiments of FIGS. 1-10 and fixes the adjacent vertebral bodies together in a dynamic fashion providing immediate postoperative stability and support of the spine. The dynamic rod assembly permits relative movement of the first and second rod portions 12, 14. The dynamic rod allows for polyaxial movement described by a displacement from the longitudinal axis as well as movement along the longitudinal axis alone or in combination allowing the rod to carry some of the natural flexion and extension moments that the spine is subjected to.

In one variation, the bias element 16 is a compression spring that becomes shorter when axially loaded under a compression force and acts as an extension mechanism such that when disposed in the assembled dynamic rod 10 and longitudinally loaded into compression, the bias element 16 exerts a biasing force moving the second rod portion 14 and retainer 17 apart. When extended beyond the static “relaxed” length, the bias element 16 exerts a biasing force towards the “relaxed” length position. Such a configuration advantageously tends to return a contraction or extension of the rod to a normal elongated “relaxed” position. In this variation, the dynamic rod 10 under extension load is advantageously permitted to elongate; and when elongated, the bias element 16 urges the rod 10 back to its contracted “relaxed” length thereby biasing the elongation inwardly.

In another variation, the bias element 16 is a tension spring that becomes longer when axially loaded under an extension force and acts as a contraction mechanism such that when disposed in the assembled dynamic rod 10 and axially loaded, the bias element 16 exerts a biasing force pulling the second rod portion 12 and the retainer 17 together. The tension spring is incapable of being compressed due to its static closely coiled length. In this variation, the dynamic rod 10 under a load that extends the bias element 16; and when extended, the bias element 16 urges the second rod portion 14 and retainer 17 together.

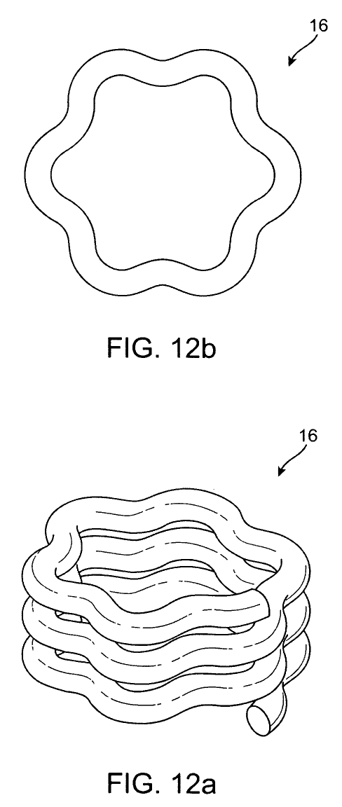

Turning now to FIGS. 12a and 12b, there is shown a variation of the bias element 16 according to the present invention. In this variation, the bias element 16 is a spring having a corrugated shape as seen in the top planar view of FIG. 12b. The corrugated bias element 16 permits closer contact with the central portion of the second rod portion 14 at corrugated sections of the spring that fold inwardly as well as closer contact with the sidewalls of the receiving portion 30 at corrugated sections of the spring that fold outwardly. As a result, the corrugated bias element advantageously provides greater stability and support of the second rod portion 14 while disposed within the receiving portion 30 of the first rod portion 12 as it limits the degree of displacement from the longitudinal axis of the second rod portion 14.

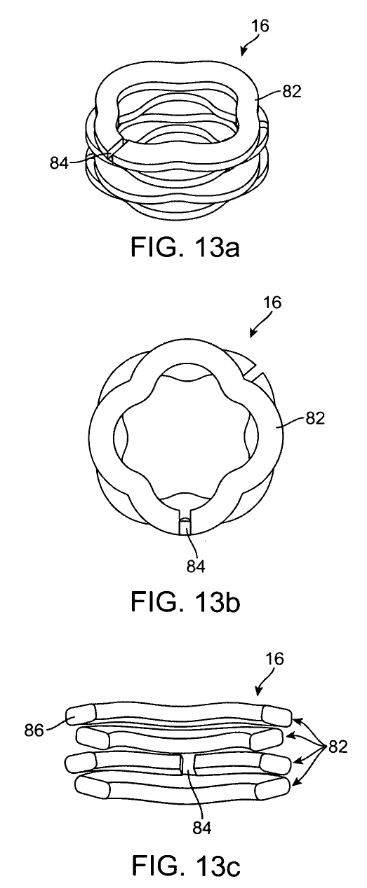

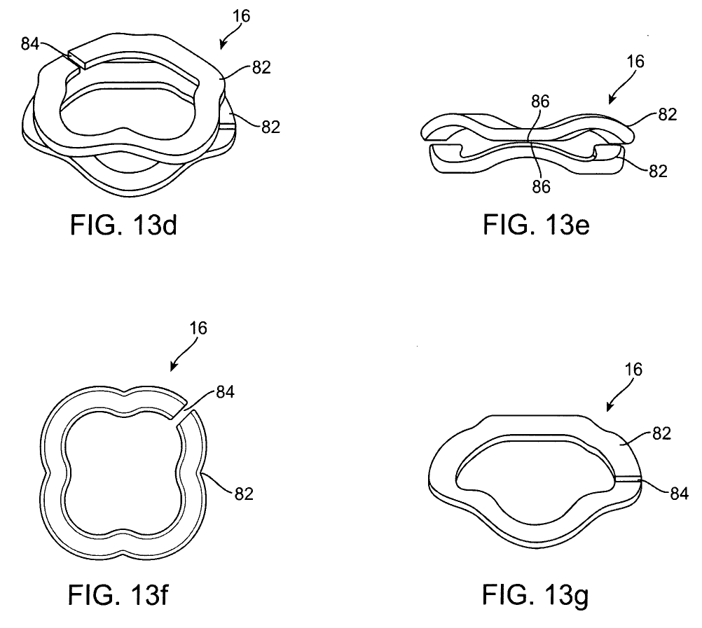

Turning now to FIGS. 13a to 13d, there is shown another variation of the bias element 16 according to the present invention. In this variation, the bias element 16 comprises at least one encompassing component 82. FIGS. 13a, 13b and 13c show four encompassing components 82 stacked together; however, the invention is not so limited and any suitable number of encompassing components may be employed. In one variation, the encompassing components 82 are rings that may or may not be corrugated. In another variation, the components 82 have distinctive sides such that the component substantially forms a square or other polygonal-like shape as shown in FIG. 13f. The component 82 may be arcuate in one variation and substantially polygonal in another variation. Any suitable shape is possible for the encompassing component 82 so long as it substantially encompasses the second rod portion 14 providing a buffer zone between the sidewalls of the receiving portion 30 and the second rod portion 14. In one variation, the at least one encompassing component 82 includes an opening 84. The opening imparts to the encompassing element 82 spring-like characteristics such that displacement of the second rod portion 14 from the longitudinal axis is biased in a substantially opposite direction by the at least one encompassing element 82. Furthermore, in another variation, the encompassing elements 82 are stacked in a staggered fashion such that the encompassing elements 82 are not aligned but turned to create a displacement relative to the adjacent elements 82 which can be seen in the top view of FIG. 13b. In yet another variation, the at least one encompassing element is substantially flat; however, the invention is not so limited and in another variation, the encompassing elements 82 are not flat. The non-flat profile imparts the encompassing element 82 with spring-like characteristics. In another variation, the non-flat profile of encompassing elements 82 form landings 86 for contacting and stacking with adjacent elements 82 as seen in cross-sectional views of FIGS. 13e and 13c. Also, the landings 86 create a displacement between stacked encompassing elements 82 and as a result, the stack of encompassing elements 82 in combination with each other form a spring. Generally, the shape of the bias element 16 as a result of the arrangement of individual encompassing elements 82 permits closer contact with the central portion of the second rod portion 14 as well as closer contact with the sidewalls of the receiving portion 30. As a result, the bias element 16 advantageously provides greater stability and support of the second rod portion 14 while disposed within the receiving portion 30 of the first rod portion 12 as it limits the degree of displacement from the longitudinal axis of the second rod portion 14 with the displacement from the longitudinal as well as displacement along the longitudinal axis being biased by the bias element 16.

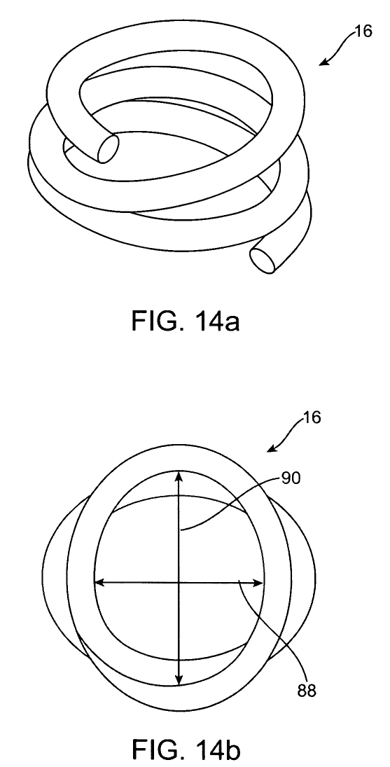

Turning now to FIGS. 14a and 14b, there is shown another variation of the bias element 16 according to the present invention. In this variation, the bias element 16 is a spring having an ellipsoidal shape as seen in the top planar view of FIG. 14b. In one variation, the bias element is configured such that when viewed from the top, the adjacent elliptical shapes are not aligned but displaced by approximately 90 degrees such that the major axis of one ellipse is approximately perpendicular to the major axis of an adjacent ellipse. In other variations, the degree of displacement may vary. The ellipsoidal bias element 16 permits closer contact with the central portion of the second rod portion 14 at minor axes sections 88 of the spring as well as closer contact with the sidewalls of the receiving portion 30 at major axes sections 90. As a result, the ellipsoidal bias element 16 advantageously provides greater stability and support of the second rod portion 14 while disposed within the receiving portion 30 of the first rod portion 12 as it limits the degree of displacement from the longitudinal axis of the second rod portion 14 and all the while providing bias along the longitudinal axis as well.

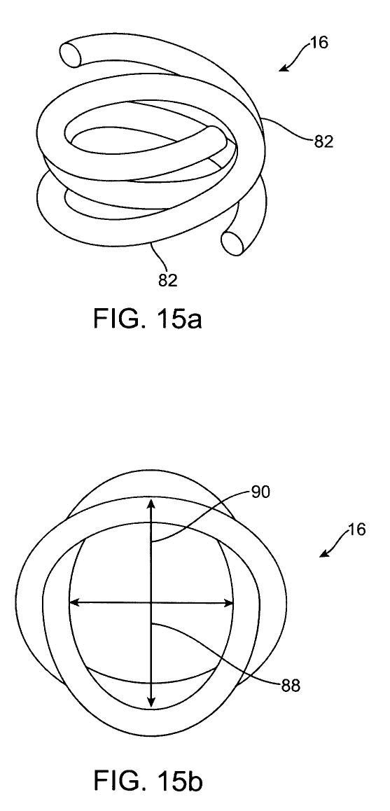

Turning now to FIGS. 15a and 15b, there is shown another variation of the bias element 16 according to the present invention. In this variation, the bias element 16 comprises at least one encompassing component 82. FIGS. 15a and 15b show two encompassing components 82 interconnected together; however, the invention is not so limited and any suitable number of encompassing components may be employed. In one variation, the encompassing components 82 are springs or coils. In another variation, the at least one encompassing component 82 is a spring having an ellipsoidal shape as clearly seen in the top planar view of FIG. 15b. The bias element 16 is configured such that the encompassing elements 82 are interconnected such that when viewed from the top, the adjacent elliptical shapes are not aligned but displaced. In one variation, the displacement is approximately 90 degrees such that the major axis of one encompassing element is approximately perpendicular to the major axis of another; however, the invention is not so limited and any suitable displacement may be employed and be dependent upon the number of encompassing elements 82 in the construct. The ellipsoidal bias element 16 permits closer contact with the central portion of the second rod portion 14 at minor axes sections 88 of the spring as well as closer contact with the sidewalls of the receiving portion 30 at major axes sections 90. As a result, the ellipsoidal bias element 16 advantageously provides greater stability and support of the second rod portion 14 while disposed within the receiving portion 30 of the first rod portion 12 as it limits the degree of displacement from the longitudinal axis of the second rod portion 14 and all the while providing bias along the longitudinal axis as well.

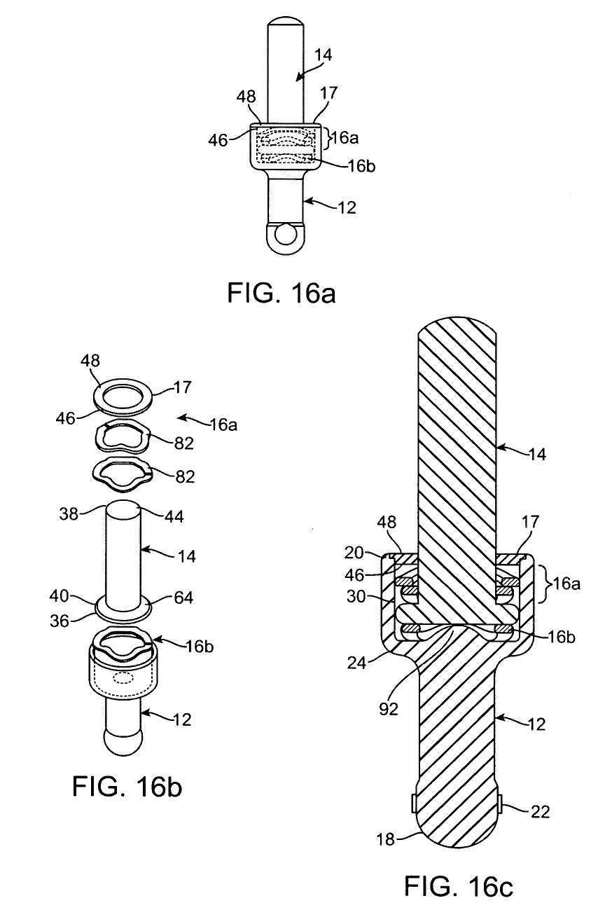

Another dynamic rod 10 according to the present invention is shown in FIGS. 16a, 16b and 16c wherein like numbers are used to describe like parts herein. In this variation, the dynamic rod 10 includes a first rod portion 12, second rod portion 14, at least one bias element 16, and a retainer 17 or other connecting means. The first rod portion 12 is connected to the second rod portion 14 via the retainer 17 and the at least one bias element 16 is disposed between the first and second rod portions 12, 14.

Still referencing FIGS. 16a and 16b and with particular reference to FIG. 16c, the first rod portion 12 includes an engaging portion 24 at a slightly enlarged and second end 20. The engaging portion 24 is configured to engage the second rod portion 14 of the dynamic rod 10. The engaging portion 24 includes a first bore defining a receiving portion 30 for receiving the second rod portion 14. The receiving portion 30 is shaped to receive a portion of the second rod portion 14 received therein. In this variation, the receiving portion 30 includes a raised portion 92 formed in the end wall of the bore of the receiving portion 30 configured to serve as a contact for the second rod portion 14. In one variation, the receiving portion 30 is configured to serve as a pivot location for the second rod portion 14 allowing it to rotate polyaxially. The raised portion 92 in one variation is centrally located in the end wall and is substantially semi-spherical in shape. However, the invention is not so limited and the raised portion 92 may be any suitable shape. The second end 20 is configured to mate with the retainer 17. The first end 18 of the first rod portion 12 includes an anchor connecting portion 22 configured to be connected to an anchor. The anchor connecting portion 22 is sized and configured to be seated in a channel of a seat of a bone screw anchor for example. Any configuration for the second end 18 that is suitable for connection to an anchor is within the scope of the present invention and, for example, may include a pin-and-slot or other configuration such as that shown in FIG. 16c for the anchor connecting portion 22 of the first rod portion 12.

With particular reference to FIG. 16b, the second rod portion 14 includes a first end 36 and a second end 38. The second rod portion 14 is generally cylindrical, elongate and rod-like in shape and includes an engaging portion 40 at an enlarged first end 36. The engaging portion 40 is configured to engage with the first rod portion 12 of the dynamic rod 10. The engaging portion 40 of the second rod portion 14 is configured and sized to fit within the receiving portion 30 of the first rod portion 14 as shown in FIGS. 16a and 16c. In this variation, the engaging portion 40 includes an encompassing shoulder or flange 64 that extends outwardly from at least a portion of the second rod portion 14. The shoulder 64 is configured as an abutment for the at least one bias element 16. The outer surface of the engaging portion 40 may be tapered such that the engaging portion narrows towards the first and or second end 36, 38. In one variation, the slope of the outer surface is approximately three degrees with respect to the longitudinal axis of the second rod portion 14; however, the invention is not so limited and the slope can is selected for customizing the angulation of the second rod portion 14 relative to the first rod portion 12. In addition, a bevel may be formed on the engaging portion 40 located closer to the second end 38. Both the taper and the bevel play a role in permitting the second rod portion 14 to angulate with respect to the first rod portion 12 when disposed inside the receiving portion 30.

Still referencing FIG. 16b, the second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, includes the pin-and-slot style configuration as shown with respect to the first rod portion 12 and discussed above.

Still referencing FIGS. 16a, 16b and 16c, the at least one bias element 16 is made from any suitable material such as titanium or PEEK. The bias element 16 is sized to be received inside the rod receiving portion 30 of the first rod portion 12. In the variation shown in FIGS. 16a, 16b and 16c, there is shown two bias elements 16a and 16b. The first bias element 16a is configured to encompass the second rod portion 14 and is disposed between the retainer 17 and the flange 64. Any type of bias element may be employed for the first bias element 16a. The first bias element 16a is a bias element comprised of two encompassing elements 82 such as those described above. A second bias element 16b is shown in FIGS. 16a, 16b and 16c. In one variation, the second bias element 16b is not employed. The second bias element 16b is configured to encompass the raised portion 92 and is disposed between the end wall of the receiving portion 30 and the flange 64 of the second rod portion 14. Any type of bias element may be employed for the second bias element 16b including any of those described herein with respect to other embodiments. In the variation shown in FIGS. 16a, 16b and 16c, the second bias element 16b is a bias element comprised of one encompassing element 82 such as any one type of the encompassing elements described above.

Still referencing FIGS. 16a, 16b and 16c, there is shown a retainer 17 having a first end 46 and a second 48 according to the present invention. In one variation, the retainer 17 is disc-like in shape and has a central bore opening to and extending between the first and second ends 46, 48 to allow passage for the central portion of the second portion 14 of the dynamic rod 10. The retainer 17 is configured to encompass at least a portion of the second rod portion 14. To capture the engaging portion 40, the retainer 17 forms a constriction such that the second end 20 has a smaller diameter opening relative to without the retainer 17 thereby at least partially closing the bore opening at the second end 20 of the first rod portion 12. The first end 46 of the retainer 17 serves as an abutment for the first bias element 16a.

Still referencing both FIGS. 16a, 16b and 16c, the assembly of the dynamic rod 10 will now be discussed. The second bias element 16b is placed inside the receiving portion 30 such that it encompasses the raised portion 92. The first bias element 16a is placed around the central portion of the second rod portion 14 such that it abuts the shoulder 64. The second rod portion 14 together with the first bias element 16a is inserted into the receiving portion 30 of the first rod portion 12. The retainer 17 is passed over the second end 38 of the second rod portion 14 such that the bore of the retainer 17 receives the central portion of the second rod portion 14. The retainer 17 is connected by laser weld or other suitable attachment means to the first rod portion 12 at the second end 20 capturing the engaging portion 40 inside the receiving portion 30 with the first bias element 16a disposed between the retainer 17 and shoulder 64 and the second bias element 16b disposed between the end wall of the receiving portion 30 and the shoulder 64.

The engaging portion 40 of the second rod portion 14 is captured by the retainer 17 and within the retainer receiving portion 30 of the first rod portion 12 such that the second rod portion 14 is capable of movement relative to the retainer 17 and the first rod portion 12. In particular, the second rod portion 14 is capable of rotation about the longitudinal axis, displacement from the longitudinal axis and/or movement along the longitudinal axis relative to the retainer 17 and the first end portion 12, such movement being biased by the first and second bias elements 16a, 16b. The movement of the second rod portion 14 relative to the first rod portion 12 is polyaxial within the constraints of the receiving portion 30. When in contact therewith, the raised portion 92 provides a contact point for such polyaxial movement of the second rod portion 14 as well as a stop limit for movement along the longitudinal axis.

The dynamic rod 10 of FIGS. 16a, 16b and 16c is implanted into the patient in the same manner as described above and fixes the adjacent vertebral bodies together in a dynamic fashion. The dynamic rod assembly permits relative movement of the first and second rod portions 12, 14 providing immediate postoperative stability and support of the spine. The dynamic rod allows for movement described by a rotation, a displacement from the longitudinal axis as well as movement along the longitudinal axis alone or in combination allowing the rod to carry some of the natural flexion and extension moments that the spine is subjected to.

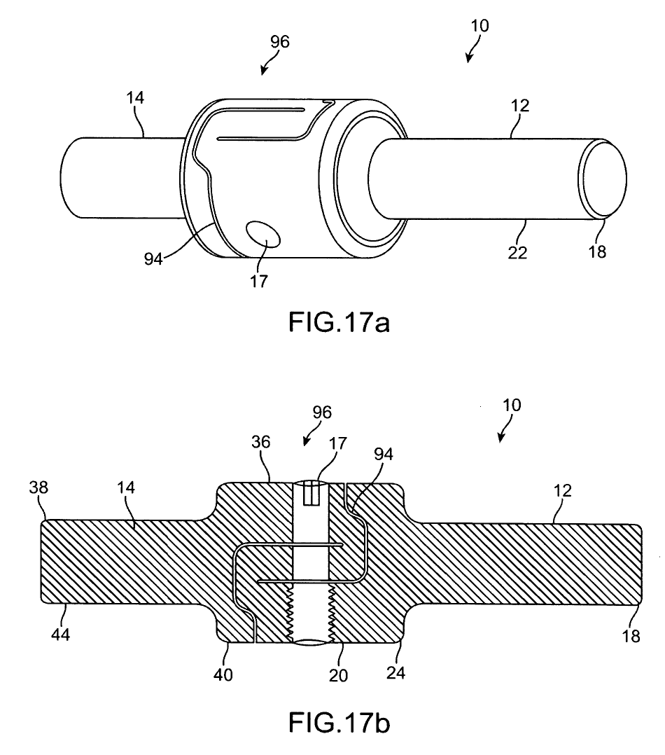

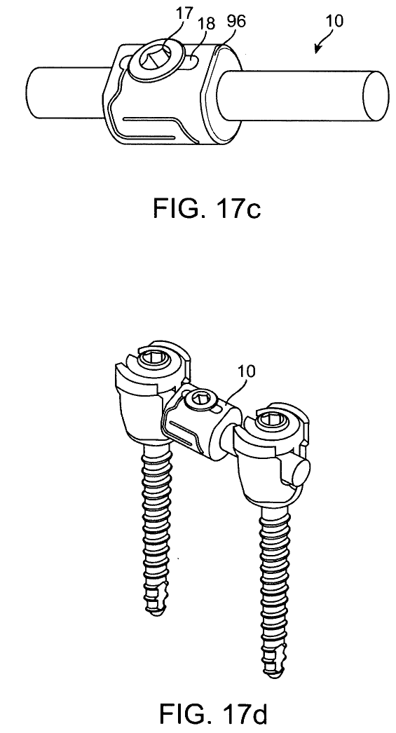

Turning now to FIGS. 17a and 17b, there is shown another variation of the dynamic rod 10 according to the invention wherein like numerals are used to describe like parts. In this variation, the dynamic rod 10 includes a first rod portion 12, second rod portion 14, and a retainer 17 or other connecting means. The first rod portion 12 is connected to the second rod portion 14 via the retainer 17.

Still referencing FIGS. 17a and 17b, the first rod portion 12 includes an engaging portion 24 at a slightly enlarged and bulbous second end 20. The engaging portion 24 is configured to engage the second rod portion 14 of the dynamic rod 10. The engaging portion 24 includes a surface that is complementary to the surface of the second rod portion 14. The engaging portion 24 can be described as comprising overlapping folds configured for interdigitation with complementary overlapping folds of the second rod portion 14. The first end 18 of the first rod portion 12 includes an anchor connecting portion 22 configured to be connected to an anchor. The anchor connecting portion 22 is sized and configured to be seated in a channel of a seat of a bone screw anchor for example. Any configuration for the second end 18 that is suitable for connection to an anchor is within the scope of the present invention and, for example, may include a pin-and-slot or other configuration for the anchor connecting portion 22 of the first rod portion 12.

Still referencing FIGS. 17a and 17b, the second rod portion 14 includes a first end 36 and a second end 38. The second rod portion 14 is generally cylindrical, elongate and rod-like in shape and includes an engaging portion 40 at an enlarged first end 36. The engaging portion 40 is configured to engage with the first rod portion 12 of the dynamic rod 10. The engaging portion 40 includes a surface that is complementary to the surface of the first rod portion 12. The engaging portion 40 can be described as comprising overlapping folds configured for interdigitation with complementary overlapping folds of the first rod portion 12. The second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, includes the pin-and-slot style configuration as shown and described above.

Still referencing FIGS. 17a and 17b, the retainer 17 comprises a screw for threading the two rod portions 12, 14 together. The engaging portions 24, 40 and the retainer 17 are made from any suitable material such as titanium or PEEK.

Still referencing both FIGS. 17a and 17b, the assembly of the dynamic rod 10 will now be discussed. Engaging portion 24 of the first rod portion 12 is connected to the engaging portion 40 by interdigitating the overlapping folds of each engaging portion 24, 40. The retainer 17 is then passed through the engaging portion to secure them together.

In another variation, the dynamic rod 10 of FIGS. 17a and 17b is not comprised of two separable elements, namely the first rod portion 12 and the second rod portion 14. Instead, the dynamic rod 12 is integrally formed such that at least one slit 94 is formed in the central section 96 that constitutes engaging portions 24, 40 of the non-integral variation. The at least one slit 94 passes through at least part of the width of the central section 96 and in one variation passes entirely through the width of the central section 96. The retainer 17 is alternatively employed to regulate and impart stiffness to the central section 96 enlivened with slits 94. The slits 94 may form any pattern and may include a snake-like pattern that creates overlapping folds or interdigitations.

With respect to any of the variations described with respect to FIGS. 17a and 17b, although there is no separate bias element in these variations of the dynamic rod 10, the biasing feature is integrally configured within the design of the central portion 96 and engaging portions 24, 40 such that flexion of the dynamic rod is permitted at these locations allowing the first rod portion 12 to deflect slightly away from the longitudinal axis. Allowing displacement of one rod portion with respect to the other rod portion in a direction along the longitudinal axis is permitted by creating a slot 98 in the central section 96 and engaging portions 24, 40 for the retainer 17 to travel within as shown in FIG. 17c. The longitudinal extension and contraction of the dynamic rod 10 is adjustable by the retainer 17 such as a screw. FIG. 17d illustrates the dynamic rod 10 of FIGS. 17a-17c deployed within two anchors.

The dynamic rod 10 of FIGS. 17a to 17d is implanted into the patient in the same manner as described above and fixes the adjacent vertebral bodies together in a dynamic fashion. The dynamic rod assembly permits relative movement of the first and second rod portions 12, 14 providing immediate postoperative stability and support of the spine. The dynamic rod allows for movement described by a displacement from the longitudinal axis as well as movement along the longitudinal axis alone or in combination allowing the rod to carry some of the natural flexion and extension moments that the spine is subjected to.

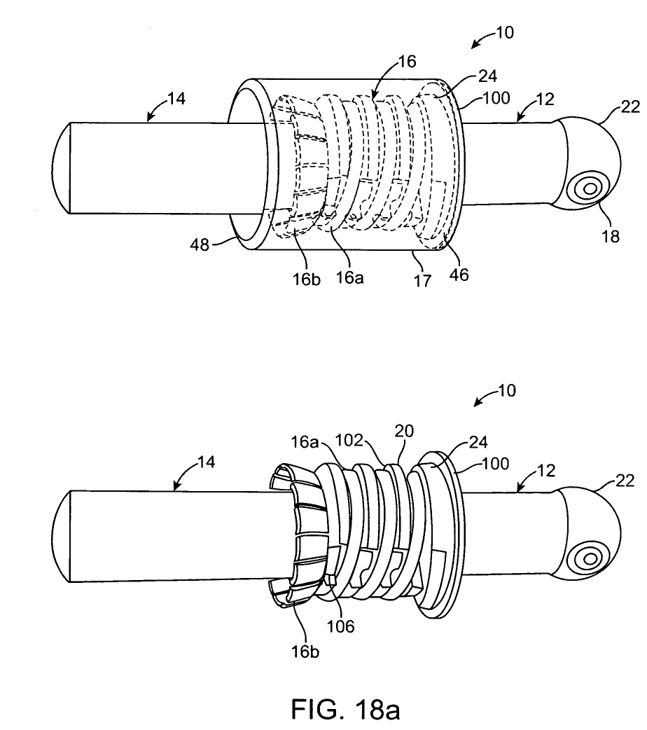

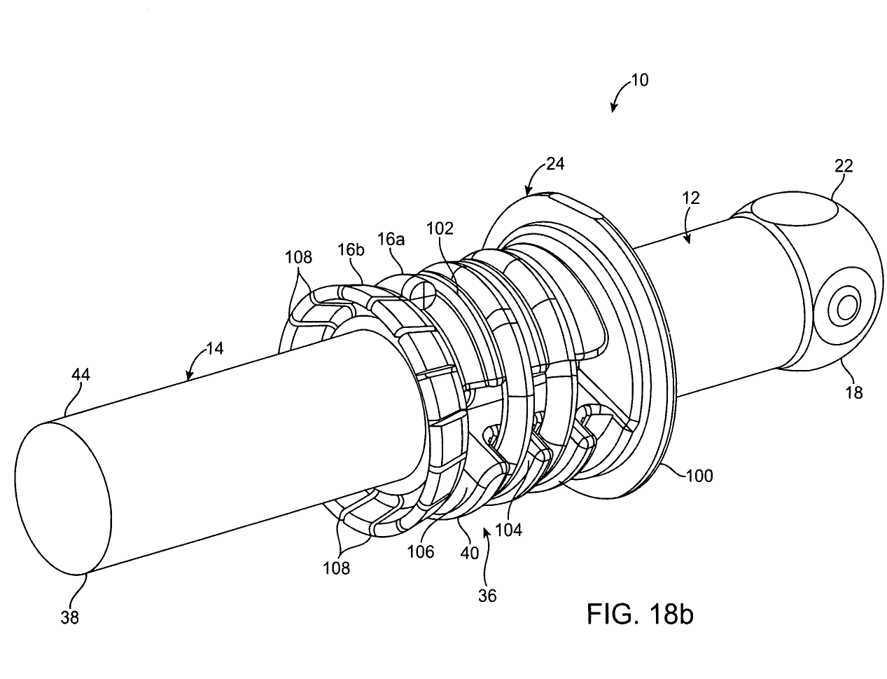

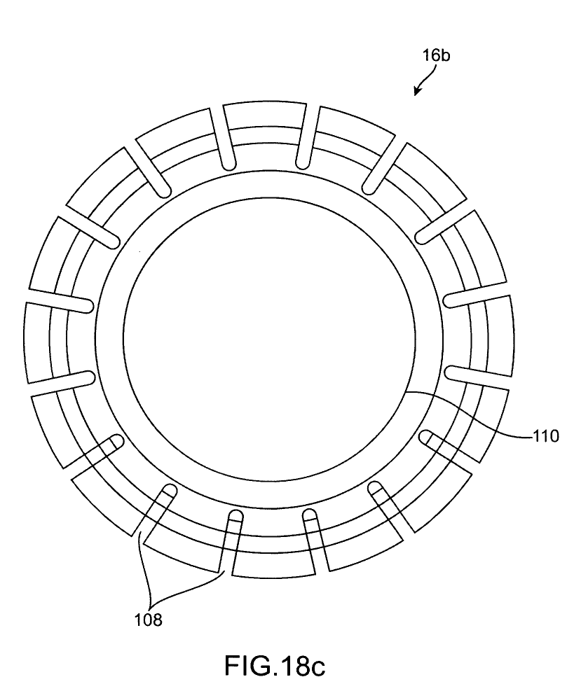

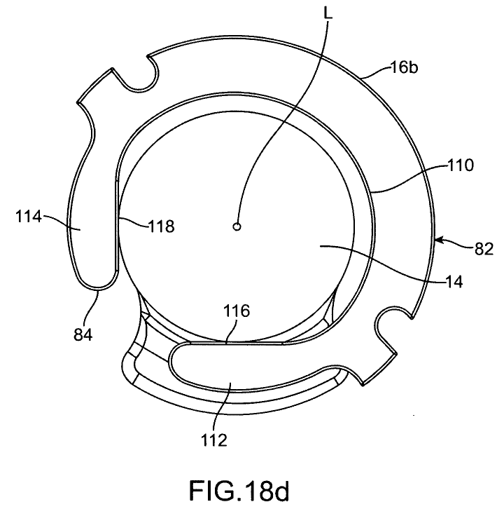

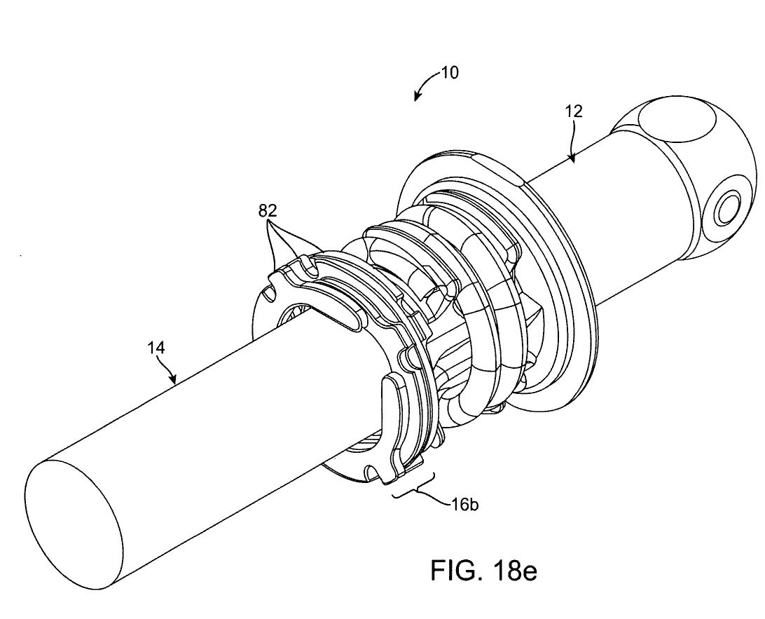

Another dynamic rod 10 according to the present invention is shown in FIGS. 18a and 18b wherein like numbers are used to describe like parts herein. In this variation, the dynamic rod 10 includes a first rod portion 12, second rod portion 14, at least one bias element 16, and a retainer 17 or other connecting means. In particular, the variation shown in FIGS. 18a and 18b include a first bias element 16a and a second bias element 16b. The first rod portion 12 is connected to the second rod portion 14 via the retainer 17 and the first bias element 16a which is disposed around at least a portion of the first and second rod portions 12, 14. The second bias element 16b is disposed around at least one of the first or second rod portions 12, 14.

Still referencing FIGS. 18a and 18b, the first rod portion 12 includes an engaging portion 24 at a slightly enlarged second end 20. The engaging portion 24 is configured to engage the second rod portion 14 of the dynamic rod 10 in a complementary fashion. The engaging portion 24 has a shape that is complementary to at least a portion of the second rod portion 14. For example, in one variation, the complementary shape is substantially a section of a cylinder such as a half cylinder that would be complementary to a half-cylinder shape of the second rod portion 14. The engaging portion 24 also includes surface features configured to receive the first bias element 16a. In the variation where the first bias element 16a is a coil, the surface features 102 include thread-like grooves for receiving at least a portion of the coil therein. The engaging portion 24 includes a flange 100. The first end 18 of the first rod portion 12 includes an anchor connecting portion 22 configured to be connected to an anchor. The anchor connecting portion 22 is sized and configured to be seated in a channel of a seat of a bone screw anchor for example. Any configuration for the second end 18 that is suitable for connection to an anchor is within the scope of the present invention and, for example, may include a pin-and-slot or other configuration

Still referencing FIGS. 18a and 18b, the second rod portion 14 includes a first end 36 and a second end 38. The second rod portion 14 is generally cylindrical, elongate and rod-like in shape and includes an engaging portion 40 at an enlarged first end 36. The engaging portion 40 is configured to engage with the first rod portion 12 of the dynamic rod 10 in a complementary fashion. The engaging portion 40 has a shape that is complementary to at least a portion of the first rod portion 12. For example, in one variation, the complementary shape is substantially a section of a cylinder such as a half cylinder that would be complementary to a half-cylinder shape of the first rod portion 12. The engaging portion 40 also includes surface features 104 configured to receive the first bias element 16a. In the variation where the first bias element 16a is a coil, the surface features 104 include thread-like grooves for receiving at least a portion of the coil therein. The engaging portion 40 includes a flange 106. The second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, may include a pin-and-slot or other configuration.

Still referencing FIGS. 18a and 18b, the first bias element 16a is made from any suitable material such as titanium or PEEK. The first bias element 16a is sized to encompass the engaging portions 24, 40 of the first and second rod portions 12, 14, respectively. In the variation shown in FIGS. 18a and 18b, the first bias element 16a is a coil; however, any type of bias element may be employed for the first bias element 16a including any of those described herein with respect to other embodiments.

Also shown in FIGS. 18a and 18b is a second bias element 16b. The second bias element 16b is made from any suitable material such as titanium or PEEK. In one variation, the second bias element 16b is not employed. The second bias element 16b is configured to encompass at least one of the first or second rod portions 12, 14. In FIGS. 18a and 18b, the second bias element 16b is shown to encompass a portion of the second rod portion 14 at a location just outside of the engaging portion 40 adjacent to the flange 106. In another variation, the second bias element 16b is positioned on the first rod portion 12 just outside the engaging portion 24 adjacent to the flange 100. And yet in another variation, a third bias element is provided such that the second and third bias elements are positioned on the first and second rod portions 12, 14 adjacent to flanges 100, 106.

With particular reference to FIG. 18c, the second bias element 16b is substantially circular in shape with a central aperture 110 for receiving a rod portion therein. The second bias element 16b comprises a section of a cone with a plurality of slits 108 that open at the outer periphery and extend inwardly towards the aperture 110 as shown in FIG. 18c. The slits 108 impart the second bias element 16b with spring-like characteristics such that the second bias element has potential for elastic deflection for providing a spring force when loaded.

Another variation of the second bias element 16b is shown in FIG. 18d which is a cross-sectional view of the dynamic rod assembly pictured in FIG. 18e. The second bias element 16b is substantially circular in shape with a central aperture 110 for receiving a rod portion therein. The second bias element 16 includes an opening 84 and two fingers 112, 114 positioned at opposite sides of the opening 84. The opening 84 is shown to extend from the outer periphery all the way to the aperture 110; however, the invention is not so limited and, in one variation, the opening 84 may extend partially into the bias element 16b. The opening 84 imparts the second bias element 16b with spring-like characteristics such that an annular spring is formed with the element having the potential for elastic deflection and spring response. Each finger 112, 114 is formed to slightly constrict the aperture 110 as seen in FIG. 18d. In the variation shown, each finger 112, 114 includes flat areas 116, 118. When the rod portion 14, for example, is deflected from the longitudinal axis “L”, one or both of the fingers 112, 114 contact the rod portion 14 and the contacting finger or fingers is capable of deflection relative to the rest of the bias element 16b. The fingers have a narrow width relative to the wider rest of the bias element 16b and are first to exhibit a spring response. The rest of the bias element 16b is also capable of exhibiting a spring response as discussed above. Although the second bias element 16b is described as being “second”, the invention is not so limited and the second bias element 16b being the only or first bias element is within the scope of the invention and a variation that is not depicted in the figures.

The dynamic rod assembly that includes the second bias element 16b described with respect to FIG. 18d is shown in FIG. 18e. In particular, the second bias element 16b is shown comprising more than one of the encompassing elements 82 shown and described with respect to FIG. 18d. In particular, three encompassing elements are shown in FIG. 18d, but the invention is not so limited and at least one encompassing element 82 is within the scope of the present invention. The encompassing elements 82 are placed in a staggered orientation with respect to one another around the rod portion such that the fingers are spaced around the rod portion.

With particular reference to FIG. 18a, there is shown a retainer 17 having a first end 46 and a second 48 according to the present invention. The retainer 17 is generally cylindrical in shape and has a bore opening to and extending between the first and second ends 46, 48. The retainer 17 is configured to encompass at least a portion of the first rod portion 12 and at least a portion of the second rod portion 14. The retainer 17 is made of titanium, PEEK, polyeurathane or silicone or any other suitable polymeric or metallic material. In one variation, the retainer 17 is injection molded around the dynamic rod 10 after it is assembled. The dynamic rod assemblies are shown without the retainer 17 in FIGS. 18b and 18e; however, a retainer 17 is clearly employable in those variations and is within the scope of the present invention.

Still referencing both FIGS. 18a to 18e, the assembly of the dynamic rod 10 will now be discussed. The two complementary portions of the first and second rod portions 12, 14 are connected and the first bias element 16a is placed around the engaging portions 24, 40. If the engaging portions 24, 40 include special surface features 104, then the first bias element 16a is disposed within them. The second bias element 16b is placed around one of the rod portions and the retainer 17 is disposed around the engaging portions 24, 40.

The first rod portion 12 is capable of movement relative to the second rod portion 14. In particular, the second rod portion 14 is capable of displacement from the longitudinal axis and/or movement along the longitudinal axis relative to the first rod portion 12, such movement being biased by the first and second bias elements 16a, 16b. The movement of the second rod portion 14 relative to the first rod portion 12 is substantially polyaxial within the constraints of the retainer 17.

The dynamic rod 10 of FIGS. 18a to 18e is implanted into the patient in the same manner as described above and fixes the adjacent vertebral bodies together in a dynamic fashion. The dynamic rod assembly permits relative movement of the first and second rod portions 12, 14 providing immediate postoperative stability and support of the spine. The dynamic rod allows for movement described by a displacement from the longitudinal axis as well as movement along the longitudinal axis alone or in combination allowing the rod to carry some of the natural flexion and extension moments that the spine is subjected to.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A dynamic rod comprising:
a first rod portion having a first engaging portion at one end; the first engaging portion having a second rod receiving portion configured to receive a second rod portion; the first engaging portion further having a first bias element receiving portion;
a second rod portion having a second engaging portion at one end; the second engaging portion having a second bias element receiving portion;
wherein the first and second rod portions are connected to each other at the first and second engaging portions such that at least a portion of the second engaging portion is disposed in the second rod receiving portion;
a retainer configured to keep the first and second rod portions together;
at least a first bias element configured to provide a bias force;
wherein at least a portion of the first bias element is disposed in the first bias element receiving portion and at least another portion of the first bias element is disposed in the second bias element receiving portion; and the first bias element being disposed between the first and second rod portions.

2. The dynamic rod of claim 1 wherein the first bias element receiving portion is located inside the second rod receiving portion.

3. The dynamic rod of claim 1 wherein in the retainer is configured to encompass at least a portion of the first rod portion and at least a portion of the second rod portion and connected to the first rod portion such that the second rod portion is capable of movement relative to the first rod portion.

4. The dynamic rod of claim 1 further including a stiffener located between the first and second rod portions.

5. The dynamic rod of claim 1 further including:
a second bias element;
wherein the second rod engaging portion includes a flange and the retainer includes a interior ledge;
wherein the second bias element is disposed between the flange and the ledge.

6. The dynamic rod of claim 1 wherein the bias element is configured to provide a bias force on one of the first and second rod portions relative to the other of the first and second rod portions.

7. The dynamic rod of claim 1 wherein the bias element is configured to provide a bias force on one of the first and second rod portions upon relative motion with respect to the other of the first and second rod portions.

8. A dynamic rod comprising:
a first rod portion having a first engaging portion at one end; the first engaging portion having a first bias element receiving portion;
a second rod portion having a second engaging portion at one end; the second engaging portion having a second bias element receiving portion;
wherein the first and second rod portions are connected to each other at the first and second engaging portions;
a retainer configured to keep the first and second rod portions together;
at least a first bias element configured to provide a bias force;
wherein at least a portion of the first bias element is disposed in the first bias element receiving portion and at least another portion of the first bias element is disposed in the second bias element receiving portion; the first bias element being disposed between the first and second rod portions.

9. The dynamic rod of claim 8 wherein the retainer is configured to encompass the first bias element.

10. The dynamic rod of claim 8 further including a bearing element disposed between the first and second engaging portions.

11. The dynamic rod of claim 8 wherein the first engaging portion overlaps the second engaging portion such that a cross-section of the first engaging portion taken perpendicular to the longitudinal axis of the dynamic rod is complementary to the second engaging portion at said cross-section.

12. The dynamic rod of claim 11 wherein the first and second engaging portions have thread-like grooves configured to receive a coil-like first bias element.

13. The dynamic rod of claim 11 further including at least one second bias element.

14. The dynamic rod of claim 13 wherein the second bias element is substantially circular in shape with a central aperture for receiving a rod portion therein with the first or second rod portion located in the central aperture; the second bias element further includes a plurality of slits that open at the outer periphery of the bias element and extend inwardly toward the longitudinal axis of the dynamic rod.

15. The dynamic rod of claim 13 wherein the second bias element is ring-like in shape and includes a central aperture for receiving a rod portion therein with the first or second rod portion located in the central portion and an opening in the second bias element forming two fingers that constrict the central aperture.

16. The dynamic rod of claim 8 wherein the at least a first bias element is configured to provide a bias force on one of the first and second rod portions relative to the other of the first and second rod portions.

17. The dynamic rod of claim 8 wherein the at least a first bias element is configured to provide a bias force on one of the first and second rod portions relative to the other of the first and second rod portions upon motion of one of the first and second rod portions with respect to the other one of the first and second rod portions

18. A dynamic rod comprising:
a first rod portion having a first engaging portion at one end; the first engaging portion having a second rod receiving portion configured to receive a second rod portion;
a second rod portion having a shaped second engaging portion at one end;
wherein the first and second rod portions are connected to each other at the first and second engaging portions such that the second engaging portion is disposed in the second rod receiving portion and such that the first rod portion is movable relative to the second rod portion;
a retainer configured to keep the first and second rod portions together;
at least a first bias element configured to provide a bias force;
wherein the first bias element is disposed in the second rod receiving portion between the shaped second engaging portion and the retainer.

19. The dynamic rod of claim 18 wherein the second rod receiving portion is a bore having a partially spherical shaped bottom; and wherein the second engaging portion has a partially spherical shape corresponding to the partially spherical shaped bottom such that the second engaging portion moves relative to the base to pivot the second rod portion relative to the first rod portion.

20. The dynamic rod of claim 18 wherein the second rod receiving portion is a bore having a base; the base includes a raised portion configured to contact the second engaging portion such that the second engaging portion pivots about the contact.

21. The dynamic rod of claim 20 further including a second bias element disposed between the base and the second engaging portion.

22. The dynamic rod of claim 18 wherein the bias element is configured to provide a bias force on one of the first and second rod portions with respect to the other of the first and second rod portions upon motion of one of the first and second rod portions with respect to the other one of the first and second rod portions.

23. The dynamic rod of claim 18 at least a first bias element configured to provide a bias force on one of the first and second rod portions with respect to the other of the first and second rod portions

24. A dynamic rod comprising:
a first rod portion having a first engaging portion at one end;
a second rod portion having a second engaging portion at one end;
wherein the first and second rod portions are connected to each other at the first and second engaging portions such that the first rod portion is movable relative to the second rod portion;
at least a first bias element configured to provide a bias force on one of the first and second rod portions upon relative motion with respect to the other of the first and second rod portions;
wherein at least a portion of the first bias element is disposed between the first and second rod portions;
wherein the first bias element includes a central opening and at least partially encompasses one of the first and second rod portions; the first bias element includes a radial axis that is not constant.

25. The dynamic rod of claim 24 wherein the first bias element includes a major axis and a minor axis; wherein the first bias element is closer to one of the first and second rod portions at the minor axis and closer to the other of the first and second rod portions at the major axis.

26. The dynamic rod of claim 24 wherein the non-constant radial axis forms a plurality of corrugations in the first bias element.

27. The dynamic rod of claim 24 wherein the bias element includes at least one at least partially encompassing component.

28. The dynamic rod of claim 24 wherein the encompassing component includes at least one landing perpendicular to the longitudinal axis of the dynamic rod.

29. The dynamic rod of claim 27 wherein the bias element includes a plurality of stacked encompassing components.

30. A dynamic rod comprising:
a first rod portion having a first engaging portion at one end;
a second rod portion having a second engaging portion at one end;
wherein the first and second rod portions are connected to each other at the first and second engaging portions such that the first and second engaging portions form at least one overlap configured to impart the dynamic rod with greater flexibility at intersection of the first and second engaging portions relative to the rest of the rod portions such that the first rod portion is movable relative to the second rod portion.

31. The dynamic rod of claim 30 wherein the first and second rod portions are integrally formed from the same piece.

32. The dynamic rod of claim 30 wherein the at least one overlap forms at least one interdigitation of first and second rod portions.

33. The dynamic rod of claim 30 further including a retainer configured to connect the first and second rod portions together.
